(19) United States
(12) Patent Application Publication
(10) Pub. No.: US 2009/0287103 A1
(43) Pub. Date: Nov. 19, 2009

(54) SYSTEMS AND METHODS FOR MONITORING PATIENT ACTIVITY AND/OR EXERCISE AND DISPLAYING INFORMATION ABOUT THE SAME

(75) Inventor: Ajit Pillai, Sunnyvale, CA (US)

Correspondence Address: STEVEN M MITCHELL;PACESETTER INC, 701 EAST EVELYN AVENUE, SUNNYVALE, CA, 94086, US

(73) Assignee: PACESETTER, INC., Sunnyvale, CA (US)

(21) Appl. No.: 12/120,499

(22) Filed: May 14, 2008

(51) Int. Cl. A61B 5/04; A61N 1/00

(52) U.S. Cl. 600/509; 607/19; 607/17

(57) ABSTRACT

Patient activity and heart rate (HR) are monitored. For each of a plurality of time periods, periods of patient exercise and/or patient activity, if any, are detected based on the monitored patient activity and HR and an activity threshold. A cumulative duration of exercise and/or a cumulative duration of activity is/are determined for each time period, and the peak exercise HR for each period of patient exercise is detected. Information is stored, including duration information indicative of the cumulative duration of exercise and/or the cumulative duration of activity for each time period, and peak exercise information associated with the period of patient exercise during which the highest peak exercise HR occurred for each time period. The stored duration information, or information indicative thereof, are transferred from an implantable system to a non-implanted system so that a graphical representation of such information can be displayed and observed to evaluate exercise compliance and/or heart failure condition.

### RELATED APPLICATIONS

The present application is related to the following commonly assigned applications, which are each incorporated herein by reference: U.S. patent application Ser. No. 10/828,897, filed Apr. 20, 2004, entitled “Methods And Devices For Determining Heart Rate Recovery,” now U.S. Pat. No. 7,043,294; U.S. patent application Ser. No. 11/405,129, filed Apr. 13, 2006, entitled “Methods and Devices For Determining Exercise Compliance Diagnostics”; and U.S. patent application Ser. No. 11/458,614, filed Jul. 19, 2006, entitled “Methods and Systems For Optimizing Exercise Compliance Diagnostic Parameters”; and U.S. patent application Ser. No. 11/561,267, filed Nov. 17, 2006, entitled “Methods and Devices for Monitoring Exercise Activity”.

### FIELD OF THE INVENTION

Embodiments of the present invention relate generally to implantable cardiac devices and, more particularly, to implantable cardiac devices that can monitor patient exercise and/or activity. Embodiments of the present invention also relate to non-implantable systems that can obtain information about patient exercise and/or activity from implantable cardiac devices, and can display such information in a useful manner.

### BACKGROUND

An implantable cardiac device is a medical device that is implanted in a patient to monitor electrical activity of the heart and to deliver appropriate electrical and/or drug therapy, as required. Implantable cardiac devices include, for example, pacemakers, cardioverters and defibrillators. The term “implantable cardioverter defibrillator” or simply “ICD” is used herein to refer to any implantable cardiac device. An ICD employs a battery to power its internal circuitry and to generate electrical therapy. The electrical therapy can include, for example, pacing pulses, cardioverting pulses and/or defibrillator pulses.

Heart failure is a growing medical challenge. In clinical practice today, most patients are managed effectively through pharmacological therapy such as beta-blockers, ACE inhibitors, and diuretics. If a patient's condition worsens, treatment may become more aggressive to include biventricular pacing and other implantable cardiac device therapy. Along with providing the primary objectives in the treatment of heart failure of improving symptoms, increasing the quality of life, and slowing disease progression, it is desirable that devices provide heart failure physicians with diagnostic parameters to monitor the patient's progress.

Currently, medical history and physical examination are the most important tools that a physician uses to determine and mark the progress of a heart failure patient. This involves much of the physician's time with the patient, as this may lead to the primary management program for the patient.

Included in most management programs is an exercise routine. It has been written extensively that adherence to exercise is a priority in improving or in maintaining good heath. Exercise diagnostics may help clinicians assess the compliance of the management programs prescribed to their patients, and possibly assist the patient in meeting those goals.

During exercise, the heart rate is a parameter or indicator of the amount of work that was required to provide blood and oxygen to the body. The maximum heart rate for a level of exercise corresponds to the conditioning of the heart. Other parameters, such as heart rate intensity, percent oxygen consumption (% VO₂) reserve, metabolic equivalents (METS), and workload also provide data that is indicative of heart conditioning.

Heart rate recovery after exercise is evaluated as a clinical marker of good vagal activity and cardiac health. As the heart rate increases due to a reduction in vagal tone, the heart rate also decreases with a reactivation of vagal activity. A delayed response to the decreasing heart rate may be a good prognostic marker of overall mortality (Cole, C. et al., NEJM 341:18, 1351-1357 (1999)) and cardiac health. Cole suggests that a reduction of only 12 beats per minute after one minute from peak exercise has been shown to be an abnormal value.

As previously mentioned, adherence to an exercise routine is a priority in managing heart failure progression; therefore, it is critical that a physician monitor patient activity, i.e., the time the patient is moving around, and patient exercise, i.e., the time the patient is continuously moving around. One method of monitoring patient activity and exercise relies on subjective and often inaccurate reporting of exercise duration and workload/intensity level by the patient.

Other more objective methods rely on algorithms that monitor patient activity using physiological sensors. Such algorithms use a single, fixed activity-sensor exercise threshold to detect exercise duration. However, current sensors in use are one dimensional, in that they only detect patient movement in one direction, depending on there orientation with respect to the patient. For example, a sensor may be oriented such that it senses movement only in the horizontal, forward-backward direction. As a result, exercise formats involving vertically directed activity, such as climbing stairs or exercise such as biking cannot be easily detected. Additionally, each patient has different movement style—some may breathe hard enough to cause some sensor counts, and a single exercise threshold for all the patients may produce inaccurate results.

It would be useful if systems, methods and devices were available that help a physician or other user objectively evaluate exercise compliance and heart failure progression (or regression) using current (or future developed) sensor technology.

### SUMMARY

Embodiments of the present invention relate to methods, systems and devices for patient monitoring. In accordance with an embodiment, an implantable system is used to perform the following, for each of a plurality of time periods (e.g., for each day, of 60 days). Patient activity and heart rate (HR) are monitored. Periods of patient exercise and/or patient activity, if any, are detected based on the monitored patient activity and HR and an activity threshold. A cumulative duration of exercise and/or a cumulative duration of activity is/are determined for each time period (e.g., each day), and the peak exercise HR for each period of patient exercise is detected. Information is stored, including duration information indicative of the cumulative duration of exercise and/or the cumulative duration of activity for each time period, and peak exercise information associated with the period of patient exercise during which the highest peak exercise HR occurred for each time period (e.g., such peak exercise information can include HR information). Additionally, or alternatively, heart rate recovery information can be stored for each time period. The stored duration information, peak exercise information, and/or the stored heart rate recovery information, or information indicative thereof, are transferred from the implantable system to a non-implanted system so that a graphical representation of such information can be displayed in a useful manner.

In accordance with an embodiment, a graph that shows the cumulative duration of patient exercise for the plurality of time periods is displayed. Additionally or alternatively, a graph can show the cumulative duration of patient activity for the plurality of time periods. Further, in response to a user selecting one of the plurality of time periods represented in a graph, a further graph can be displayed that shows HR information for the period of patient exercise that includes the peak patient exercise HR for the selected time period. Such a further graph can include, e.g., HRs preceding and following the peak exercise HR.

In accordance with an embodiment, in response to a user selecting one of the plurality of time periods, a numerical value indicative of the cumulative duration of patient exercise and/or the cumulative duration of patient activity is displayed for the selected time period. In accordance with an embodiment, in response to a user selecting one of the plurality of time periods, a numerical value indicative of the peak exercise HR and/or heart rate recovery is displayed for the selected time period. These and other aspects and advantages of embodiments of the invention will become apparent from the following detailed description and the accompanying drawings which illustrate by way of example the features of embodiments of the invention.

Further and alternative embodiments, and the features, aspects, and advantages of the embodiments of invention will become more apparent from the detailed description set forth below, the drawings and the claims.

### BRIEF DESCRIPTION OF THE DRAWINGS

FIG. 1A is a simplified diagram illustrating an exemplary ICD in electrical communication with at least three leads implanted into a patient's heart for delivering multi-chamber stimulation and shock therapy.

FIG. 1B is a functional block diagram of an exemplary ICD, which can provide cardioversion, defibrillation and pacing stimulation in four chambers of a heart.

FIG. 2 is a flow chart illustrating an embodiment of a method for determining an observed maximum heart rate of a patient during exercise.

FIGS. 3A-3D illustrate representations of sample exercise diagnostic results.

FIG. 4 is a flow chart illustrating an embodiment of a method for determining an exercise diagnostic such as work of a patient during exercise.

FIG. 5 is a flow chart illustrating an embodiment of a method for determining heart rate recovery of a patient.

FIG. 6 is a flow chart illustrating another embodiment of a method for determining an observed maximum heart rate of a patient during exercise.

FIG. 7 is a flow chart illustrating another embodiment of a method for determining heart rate recovery of a patient.

FIG. 8 is a flow chart illustrating an embodiment of a method for determining patient activity duration and exercise duration.

FIG. 9 is a flow chart illustrating an embodiment of a method for determining patient-specific offset parameters used in the method of FIG. 8.

FIG. 10 is a flow chart illustrating an embodiment of a method for determining an activity threshold used in the method of FIG. 8.

FIG. 11 is an example of a histogram of activity correlation values that may be used to determine the activity threshold used in the method of FIG. 8.

FIG. 12 is an exemplary time line illustrating the start and end of activity and exercise time periods.

FIG. 13 is a flow chart illustrating an embodiment of a method for detecting exercise and/or rest of a patient based on heart rate data.

FIG. 14 is a flow chart illustrating an embodiment of a method for detecting exercise based on patient heart rate and activity data.

FIG. 15 is a flow chart illustrating an embodiment of a method for patient monitoring.

FIG. 16 is a flow chart illustrating an alternative embodiment of a method for patient monitoring.

FIG. 17 is an example of a graph that shows the cumulative duration of patient exercise for a plurality of time periods, and a graph that shows the cumulative duration of patient activity for the plurality of time periods, according to an embodiment.

FIG. 18 is an example of a graph that shows HR information for the period of patient exercise that includes the peak patient exercise HR for a selected time period, according to an embodiment.

FIG. 19 is an example of a graph that shows HR recovery information for a plurality of time periods, according to an embodiment.

### DETAILED DESCRIPTION

The following description is of the best mode presently contemplated for practicing embodiments of the invention. This description is not to be taken in a limiting sense but is made merely for the purpose of describing the general principles of embodiments of the invention. The scope of the embodiments of the invention should be ascertained with reference to the issued claims. In the description of the embodiments of the invention that follows, like numerals or reference designations will be used to refer to like parts or elements throughout.

It will be apparent to one of skill in the art that the present invention, as described below, may be implemented in many different embodiments of hardware, software, firmware, and/or the entities illustrated in the figures. Any actual software and/or hardware described herein is not meant to limit the scope of the present invention. Thus, the structure, operation and behavior of the present invention will be described with the understanding that many modifications and variations of the embodiments are possible, given the level of detail presented herein.

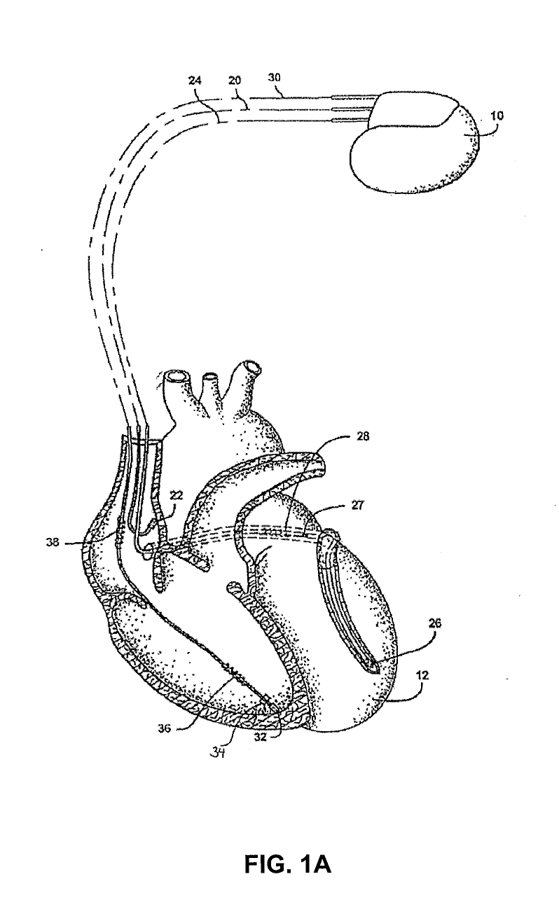

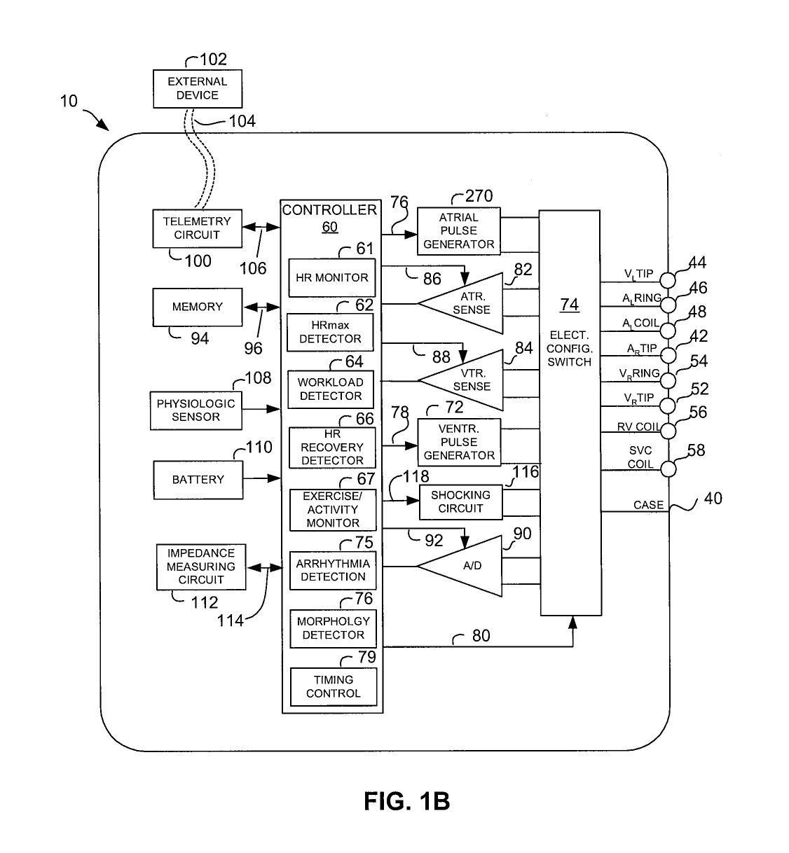

Before describing the embodiments of the invention in detail, it is helpful to describe an example environment in which embodiments of the invention may be implemented. The present invention is particularly useful in the environment of an implantable cardiac device. Implantable cardiac devices include, for example, pacemakers, cardioverter-defibrillators, and hemodynamic monitors. The term “implantable cardioverter defibrillator” or simply “ICD” is used herein to refer to any implantable cardiac device or implantable cardioverter-defibrillator. FIGS. 1A and 1B illustrate such an environment.

As shown in FIG. 1A, there is an exemplary ICD 10 in electrical communication with a patient's heart 12 by way of three leads, 20, 24 and 30, suitable for delivering multi-chamber stimulation and pacing therapy. To sense atrial cardiac signals and to provide right atrial chamber stimulation therapy, ICD 10 is coupled to implantable right atrial lead 20 having at least an atrial tip electrode 22, which typically is implanted in the patient's right atrial appendage.

To sense left atrial and ventricular cardiac signals and to provide left-chamber pacing therapy, ICD 10 is coupled to “coronary sinus” lead 24 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and shocking therapy using at least a left atrial coil electrode 28.

ICD 10 is also shown in electrical communication with the patient's heart 12 by way of an implantable right ventricular lead 30 having, in this embodiment, a right ventricular tip electrode 32, a right ventricular ring electrode 34, a right ventricular (RV) coil electrode 36, and an SVC coil electrode 38. Typically, right ventricular lead 30 is transvenously inserted into heart 12 so as to place the right ventricular tip electrode 32 in the right ventricular apex so that RV coil electrode 36 will be positioned in the right ventricle and SVC coil electrode 38 will be positioned in the superior vena cava. Accordingly, right ventricular lead 30 is capable of receiving cardiac signals and delivering stimulation in the form of pacing and shock therapy to the right ventricle.

FIG. 1B shows a simplified block diagram of ICD 10, which is capable of treating both fast and slow arrhythmias with stimulation therapy, including cardioversion, defibrillation, and pacing stimulation. While a particular multi-chamber device is shown, it is shown for illustration purposes only, and one of skill in the art could readily duplicate, eliminate or disable the appropriate circuitry in any desired combination to provide a device capable of treating the appropriate chamber(s) with the desired cardioversion, defibrillation and pacing stimulation.

A housing 40 of ICD 10, shown schematically in FIG. 1B, is often referred to as the “can,” “case” or “case electrode” and may be programmably selected to act as the return electrode for all “unipolar” modes. Housing 40 may further be used as a return electrode alone or in combination with one or more of coil electrodes, 28, 36, and 38 for shocking purposes. Housing 40 further includes a connector (not shown) having a plurality of terminals, 42, 44, 46, 48, 52, 54, 56, and 58 (shown schematically and, for convenience, the names of the electrodes to which they are connected are shown next to the terminals). As such, to achieve right atrial sensing and pacing, the connector includes at least a right atrial tip terminal (AR TIP) 42 adapted for connection to atrial tip electrode 22.

To achieve left chamber sensing, pacing and shocking, the connector includes at least a left ventricular tip terminal (VL TIP) 44, a left atrial ring terminal (AL RING) 46, and a left atrial shocking terminal (AL COIL) 48, which are adapted for connection to left ventricular ring electrode 26, left atrial tip electrode 27, and left atrial coil electrode 28, respectively.

To support right chamber sensing, pacing, and shocking the connector also includes a right ventricular tip terminal (VR TIP) 52, a right ventricular ring terminal (VR RING) 54, a right ventricular shocking terminal (RV COIL) 56, and an SVC shocking terminal (SVC COIL) 58, which are configured for connection to right ventricular tip electrode 32, right ventricular ring electrode 34, RV coil electrode 36, and SVC coil electrode 38, respectively.

At the core of ICD 10 is a programmable microcontroller 60 which controls the various modes of stimulation therapy. As is well known in the art, microcontroller 60 typically includes a microprocessor, or equivalent control circuitry, designed specifically for controlling the delivery of stimulation therapy and can further include RAM or ROM memory, logic and timing circuitry, state machine circuitry, and I/O circuitry. Typically, microcontroller 60 includes the ability to process or monitor input signals (data) as controlled by a program code stored in a designated block of memory. The details of the design of microcontroller 60 are not critical to the present invention. Rather, any suitable microcontroller 60 can be used to carry out the functions described herein. The use of microprocessor-based control circuits for performing timing and data analysis functions are well known in the art. In specific embodiments of the present invention, microcontroller 60 performs some or all of the steps associated with the exercise diagnostics in accordance with the present invention.

Representative types of control circuitry that may be used with embodiments of the invention include the microprocessor-based control system of U.S. Pat. No. 4,940,052 (Mann et al.) and the state-machines of U.S. Pat. Nos. 4,712,555 (Thornander et al.) and 4,944,298 (Sholder). For a more detailed description of the various timing intervals used within the ICDs and their inter-relationship, see U.S. Pat. No. 4,788,980 (Mann et al.). The '052, '555, '298 and '980 patents are incorporated herein by reference.

As shown in FIG. 1B, an atrial pulse generator 70 and a ventricular pulse generator 72 generate pacing stimulation pulses for delivery by right atrial lead 20, right ventricular lead 30, and/or coronary sinus lead 24 via an electrode configuration switch 74. It is understood that in order to provide stimulation therapy in each of the four chambers of the heart, atrial and ventricular pulse generators 70, 72, may include dedicated, independent pulse generators, multiplexed pulse generators, or shared pulse generators. Pulse generators 70 and 72 are controlled by microcontroller 60 via appropriate control signals 76 and 78, respectively, to trigger or inhibit the stimulation pulses.

Microcontroller 60 further includes timing control circuitry 79 which is used to control pacing parameters (e.g., the timing of stimulation pulses) as well as to keep track of the timing of refractory periods, PVARP intervals, noise detection windows, evoked response windows, alert intervals, marker channel timing, etc., which are well known in the art. Examples of pacing parameters include, but are not limited to, atrio-ventricular (AV) delay, interventricular (RV-LV) delay, atrial interconduction (A-A) delay, ventricular interconduction (V-V) delay, and pacing rate.

Switch 74 includes a plurality of switches for connecting the desired electrodes to the appropriate I/O circuits, thereby providing complete electrode programmability. Accordingly, switch 74, in response to a control signal 80 from microcontroller 60, determines the polarity of the stimulation pulses (e.g., unipolar, bipolar, combipolar, etc.) by selectively closing the appropriate combination of switches (not shown) as is known in the art.

Atrial sensing circuits 82 and ventricular sensing circuits 84 may also be selectively coupled to right atrial lead 20, coronary sinus lead 24, and right ventricular lead 30, through switch 74 for detecting the presence of cardiac activity in each of the four chambers of the heart. Accordingly, the atrial (ATR. SENSE) and ventricular (VTR. SENSE) sensing circuits 82 and 84 may include dedicated sense amplifiers, multiplexed amplifiers, or shared amplifiers. Switch 74 determines the “sensing polarity” of the cardiac signal by selectively closing the appropriate switches, as is also known in the art. In this way, the clinician may program the sensing polarity independent of the stimulation polarity.

Each sensing circuit, 82 and 84, preferably employs one or more low power, precision amplifiers with programmable gain and/or automatic gain control, bandpass filtering, and a threshold detection circuit, as known in the art, to selectively sense the cardiac signal of interest. The automatic sensitivity control enables ICD 10 to deal effectively with the difficult problem of sensing the low amplitude signal characteristics of atrial or ventricular fibrillation. Such sensing circuits, 82 and 84, can be used to determine cardiac performance values used in the present invention.

The outputs of atrial and ventricular sensing circuits 82 and 84 are connected to microcontroller 60 which, in turn, are able to trigger or inhibit atrial and ventricular pulse generators, 70 and 72, respectively, in a demand fashion in response to the absence or presence of cardiac activity, in the appropriate chambers of the heart. Sensing circuits 82 and 84, in turn, receive control signals over signal lines 86 and 88 from microcontroller 60 for purposes of measuring cardiac performance at appropriate times, and for controlling the gain, threshold, polarization charge removal circuitry (not shown), and timing of any blocking circuitry (not shown) coupled to the inputs of sensing circuits 82 and 84.

For arrhythmia detection, ICD 10 utilizes the atrial and ventricular sensing circuits 82 and 84 to sense cardiac signals to determine whether a rhythm is physiologic or pathologic. The timing intervals between sensed events (e.g., P-waves, R-waves, and depolarization signals associated with fibrillation which are sometimes referred to as “F-waves” or “Fib-waves”) are then classified by microcontroller 60 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Microcontroller 60 utilizes arrhythmia detection circuitry 75 and morphology detection circuitry 76 to recognize and classify arrhythmia so that appropriate therapy can be delivered.

Cardiac signals are also applied to the inputs of an analog-to-digital (A/D) data acquisition system 90. Data acquisition system 90 is configured to acquire intracardiac electrogram signals, convert the raw analog data into a digital signal, and store the digital signals for later processing and/or telemetric transmission to an external device 102. Data acquisition system 90 is coupled to right atrial lead 20, coronary sinus lead 24, and right ventricular lead 30 through switch 74 to sample cardiac signals across any pair of desired electrodes.

Advantageously, data acquisition system 90 can be coupled to microcontroller 60, or other detection circuitry, for detecting an evoked response from heart 12 in response to an applied stimulus, thereby aiding in the detection of “capture.” Capture occurs when an electrical stimulus applied to the heart is of sufficient energy to depolarize the cardiac tissue, thereby causing the heart muscle to contract. Microcontroller 60 detects a depolarization signal during a window following a stimulation pulse, the presence of which indicates that capture has occurred. Microcontroller 60 enables capture detection by triggering ventricular pulse generator 72 to generate a stimulation pulse, starting a capture detection window using timing control circuitry 79 within microcontroller 60, and enabling data acquisition system 90 via control signal 92 to sample the cardiac signal that falls in the capture detection window and, based on the amplitude, determines if capture has occurred.

The implementation of capture detection circuitry and algorithms are well known. See for example, U.S. Pat. No. 4,729,376 (DeCote, Jr.); U.S. Pat. No. 4,708,142 (DeCote, Jr.); U.S. Pat. No. 4,686,988 (Sholder); U.S. Pat. No. 4,969,467 (Callaghan et al.); and U.S. Pat. No. 5,350,410 (Kleks et al.), which patents are hereby incorporated herein by reference. The type of capture detection system used is not critical to the present invention.

Microcontroller 60 also contains an HR monitor 61, a maximum observed heart rate (HRₘₐₓ) detector 62, a workload detector 64, a heart rate recovery detector 66 and an exercise/activity monitor 67. The operation of the HR monitor, HRₘₐₓ detector, workload detector, heart rate recovery detector and exercise/activity monitor are discussed below in connection with the methods of the present invention. While shown as being implemented as part of the controller, the HR monitor, HRₘₐₓ detector, workload detector, heart rate recovery detector and/or exercise/activity monitor can be implemented partially or entirely separate from the controller 60, e.g., using hardware, firmware, or software, or combinations thereof.

Microcontroller 60 is further coupled to a memory 94 by a suitable data/address bus 96, wherein the programmable operating parameters used by microcontroller 60 are stored and modified, as required, in order to customize the operation of ICD 10 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 12 within each respective tier of therapy.

Advantageously, the operating parameters of ICD 10 may be non-invasively programmed into memory 94 through a telemetry circuit 100 in telemetric communication with external device 102, such as a programmer, transtelephonic transceiver, or a diagnostic system analyzer. Telemetry circuit 100 is activated by microcontroller 60 by a control signal 106. Telemetry circuit 100 advantageously allows intracardiac electrograms and status information relating to the operation of ICD 10 (as contained in microcontroller 60 or memory 94) to be sent to external device 102 through an established communication link 104.

For examples of such devices, see U.S. Pat. No. 4,809,697, entitled “Interactive Programming and Diagnostic System for use with Implantable Pacemaker” (Causey, III et al.); U.S. Pat. No. 4,944,299, entitled “High Speed Digital Telemetry System for Implantable Device” (Silvian); and U.S. Pat. No. 6,275,734, entitled “Efficient Generation of Sensing Signals in an Implantable Medical Device such as a Pacemaker or ICD” (McClure et al.), which patents are hereby incorporated herein by reference. Such devices typically include a display that will enable information collected by the ICD 10, and transferred to the external device 102, to be displayed in a useful manner, in accordance with embodiments of the present invention described in more detail below.

In one embodiment, ICD 10 further includes a physiologic sensor 108 that can be used to detect changes in cardiac performance or changes in the physiological condition of the heart. Accordingly, microcontroller 60 can respond by adjusting the various pacing parameters (such as rate, AV Delay, RV-LV Delay, V-V Delay, etc.) in accordance with the embodiments of the present invention. Microcontroller 60 controls adjustments of pacing parameters by, for example, controlling the stimulation pulses generated by the atrial and ventricular pulse generators 70 and 72. While shown as being included within ICD 10, it is to be understood that physiologic sensor 108 may also be external to ICD 10, yet still be implanted within or carried by the patient. More specifically, sensor 108 can be located inside ICD 10, on the surface of ICD 10, in a header of ICD 10, or on a lead (which can be placed inside or outside the bloodstream). As discussed below, sensor 108 can also be used to measure activity level.

ICD 10 additionally includes a battery 110 which provides operating power to all of the circuits shown in FIG. 1B. For ICD 10, which employs shocking therapy, battery 110 must be capable of operating at low current drains for long periods of time, and then be capable of providing high-current pulses (for capacitor charging) when the patient requires a shock pulse. Battery 110 must also have a predictable discharge characteristic so that elective replacement time can be detected. Accordingly, ICD 10 preferably employs lithium/silver vanadium oxide batteries, as is true for most (if not all) current devices.

ICD 10 further includes a magnet detection circuitry (not shown), coupled to microcontroller 60. It is the purpose of the magnet detection circuitry to detect when a magnet is placed over ICD 10, which magnet may be used by a clinician to perform various test functions of ICD 10 and/or to signal microcontroller 60 that the external programmer 102 is in place to receive or transmit data to microcontroller 60 through telemetry circuit 100.

As further shown in FIG. 1B, ICD 10 is shown as having an impedance measuring circuit 112 which is enabled by microcontroller 60 via a control signal 114. The known uses for an impedance measuring circuit 120 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 112 is advantageously coupled to switch 74 so that any desired electrode may be used. The impedance measuring circuit 112 is not critical to the present invention and is shown only for completeness.

In the case where ICD 10 is intended to operate as a cardioverter, pacer or defibrillator, it must detect the occurrence of an arrhythmia and automatically apply an appropriate electrical therapy to the heart aimed at terminating the detected arrhythmia. To this end, microcontroller 60 further controls a shocking circuit 116 by way of a control signal 118. The shocking circuit 116 generates shocking pulses of low (up to 0.5 Joules), moderate (0.5-10 Joules), or high energy (11 to 40 Joules), as controlled by microcontroller 60. Such shocking pulses are applied to the patient's heart 12 through at least two shocking electrodes (e.g., selected from left atrial coil electrode 28, RV coil electrode 36, and SVC coil electrode 38). As noted above, housing 40 may act as an active electrode in combination with RV electrode 36, or as part of a split electrical vector using SVC coil electrode 38 or left atrial coil electrode 28 (i.e., using the RV electrode as a common electrode).

Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 5-40 Joules), delivered asynchronously (since R-waves may be too disorganized to be recognized), and pertaining exclusively to the treatment of fibrillation. Accordingly, microcontroller 60 is capable of controlling the synchronous or asynchronous delivery of the shocking pulses.

With the description of an example environment, such as an ICD, in mind, features of embodiments of the present invention are described in more detail below. Embodiments of the present invention relate to systems and methods for monitoring patient activity and/or exercise, and displaying information about the same in a manner that is useful to an user (e.g., a doctor, clinician, etc.).

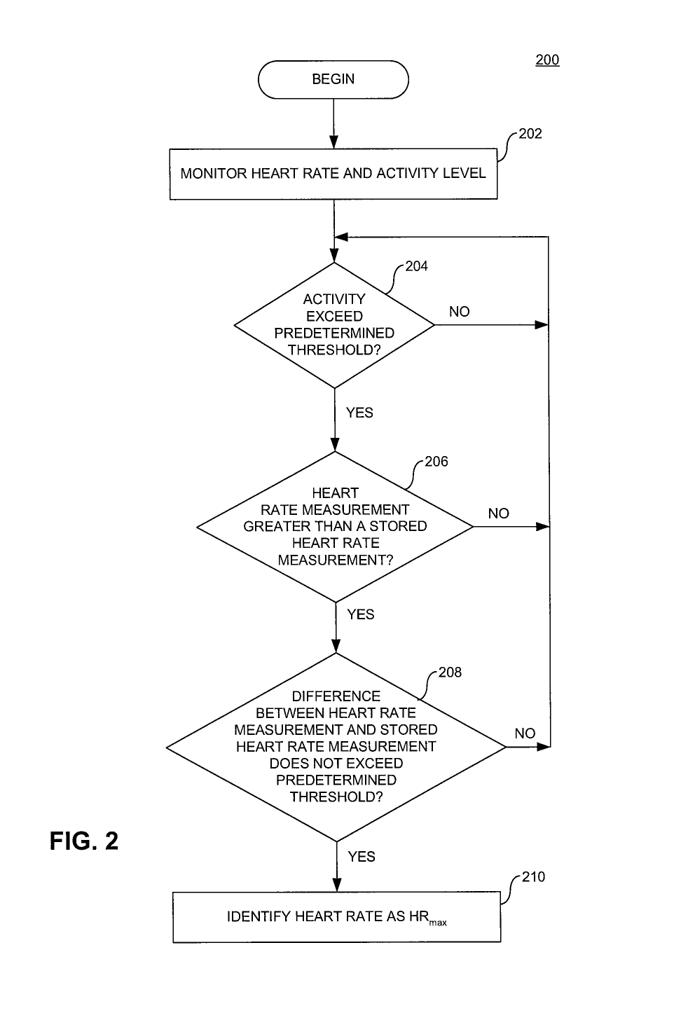

Referring to FIG. 2, a method 200 of determining a maximum observed heart rate (HRₘₐₓ) of a patient during exercise is illustrated. According to an embodiment, the method 200 begins at step 202, in which the heart rate and activity level of the patient are monitored. The heart rate and activity level of the patient may be continuously monitored during the method 200.

The patient's heart rate may be determined by any suitable method. Many variations on how to determine heart rate are known to those of ordinary skill in the art, and any of these of reasonable accuracy may be used. Heart rate can be determined by measurement of an R-R interval cycle length (or P-P), which is the inverse of heart rate. As used herein, the heart rate (in beats per minute) can be seen as the inverse to cycle length, determined by 60,000 divided by the cycle length (in milliseconds).

Heart rate measurements can be produced based upon the monitored heart rate. Such heart rate measurements include but are not limited to heart rate and heart rate intensity.

The activity level of the patient may also be determined by any suitable method. For example, the activity level may be determined by an accelerometer, piezoelectric crystal, minute ventilation, photoplethysmography, or a derivative thereof, such as the sensor indicated rate. In one embodiment, activity level is determined using physiologic sensor 108. In this embodiment, sensor 108 can be an accelerometer, a piezoelectric crystal, an impedance sensor, or a photoplethysmography sensor, but is not limited thereto.

In step 204, the measured activity level is compared with a predetermined activity threshold to determine whether the activity level exceeds the threshold. The predetermined activity threshold can be a value that corresponds to a certain level of exercise. It should be appreciated that the activity threshold value can be tailored for a specific patient's condition. Illustratively, an activity threshold value which correlates with walking or some other low level of exercise may be, for example, 50 milligravities as measured by an accelerometer.

It should be understood that in the context of embodiments of the present invention, when comparing a measurement to a threshold, the terms “exceeds” or “is greater than” can encompass instances when the measurement is equal to the threshold value. Similarly, it should be understood that the terms “falls below” or “is less than” a threshold value can encompass instances when the measurement is equal to the threshold value. A person skilled in the relevant art will recognize that selection of a threshold value, and how to treat the condition of equality between the threshold and the measurement, are design choices.

The activity level can be compared with an activity threshold at various time intervals or periodically to determine whether the activity level exceeds the predetermined threshold. The particular selected time interval for monitoring is not critical. In one embodiment of the invention, the activity level is monitored and compared with the activity threshold at time intervals of 30 seconds (i.e., every 30 seconds).

If the patient activity level exceeds the predetermined activity threshold, then the method proceeds to step 206. Illustratively, if 50 milligravities activity is a threshold that correlates well with walking or some low level of exercise and the implantable medical device is programmed at this threshold, then if the measured activity level exceeds 50 milligravities, the method proceeds to step 206.

Steps 206 and 208 can be performed when the patient activity level exceeds the predetermined activity threshold for a predetermined period of time. This predetermined period of time can be an amount that one skilled in the art would understand to be sufficient for the heart to react to the exercise by the patient (which can be indicated by, e.g., the activity level exceeding the predetermined activity threshold). Illustratively, the predetermined period of time may be 10 seconds to five minutes, preferably about two to three minutes, more preferably about two minutes.

In step 206, a heart rate measurement is compared with a stored heart rate measurement. The stored heart rate measurement can be, for example, a heart rate measurement previously obtained during exercise, including a previously determined HRₘₐₓ during exercise. Prior to first occurrence of the method, the stored heart rate measurement can be set to a predetermined default value. If the heart rate measurement exceeds the previously stored heart rate measurement, then the method proceeds to step 208. Otherwise, step 204 is repeated. That is, the method continues to monitor heart rate and activity level and produce heart rate measurements.

In step 208, the difference between the heart rate measurement and the stored heart rate measurement is compared to a predetermined threshold. The predetermined threshold difference may be selected to correspond to a value above which may be indicative of noise, PACs, PVCs, and/or arrhythmias. If the difference between the heart rate measurement and the stored heart rate measurement exceeds the threshold difference, the measured heart rate is not considered to be a HRₘₐₓ. The threshold may even be step-size units, so as to show a gradual (physiologic) increase.

In accordance with one embodiment, step 208 is not performed. However, this embodiment is less preferred, as the resulting HRₘₐₓ could be inaccurate due to noise and/or premature heartbeats.

It should be understood that the order of comparison steps 206 and 208 is not limited to that depicted in the figure and may be performed in reverse order or conducted simultaneously.

If, in step 206, the heart rate measurement is greater than the stored heart rate measurement and, in step 208, the difference between the heart rate measurement and the stored heart rate measurement does not exceed a predetermined threshold, then the heart rate associated with the heart rate measurement may be identified as a maximum observed heart rate (HRₘₐₓ) at step 210. In other words, a heart rate can be identified as a HRₘₐₓ when the comparison steps 204, 206, and 208 are met.

The maximum observed heart rate may be recorded as a stored value, and the method 200 repeated, using the HRₘₐₓ as a new stored heart rate measurement. The HRₘₐₓ determination may be continued until activity level and/or heart rate is indicative of a slow-down of exercise.

Based on the HRₘₐₓ obtained, further values may be obtained that are indicative of heart conditioning. These values include heart rate intensity, percent oxygen consumption (% VO₂) reserve, metabolic equivalents (METS), percentage METS, workload, and absolute oxygen uptake.

For example, heart rate intensity (also known as percent heart rate reserve, heart rate capacity, target heart rate, or % HRR) may be calculated by dividing HRₘₐₓ by the predicted age compensated maximum heart rate as follows:

$$\% HRR = \frac{{HR}_{max} - resting HR}{Age Compensated Maximum HR - resting HR} \times 100$$

In the equation indicated above, resting heart rate of the patient may be obtained by any suitable method including, for example, a heart rate measurement taken when the activity level of the patient is sufficiently low to be considered inactive. The age compensated maximum heart rate can be calculated by the formula: (220−age).

With % HRR, it is also possible to calculate % VO₂ reserve. Swain et al. have shown a close correlation between % HRR and % VO₂ reserve (“Heart rate reserve is equivalent to % VO₂ reserve, not % VO₂ₘₐₓ,” Med Sci. Sports Exercise 29:410-414 (1997)).

% VO₂ reserve is an intensity scale or index that describes the percentage of oxygen intake used during exercise. The value between % VO₂ reserve and 100% is the amount of oxygen intake reserves available. This value may be obtained by the following equation (Swain et al., Target HR for the development of CV fitness, Medicine & Science in Sports & Exercise 26(1):112-116):

% VO₂reserve=(% HRR−37)/0.64

where % HRR is calculated as described above.

Workload is measure of intensity times duration, and may be seen by the following equation:

Workload=Intensity*Duration

where Intensity is VO_{2observed}, but may also be seen as an index such as heart rate intensity (% HRR) or % VO₂ reserve as discussed above, and Duration is the time during exercise when activity is above a predetermined threshold. It is possible to use in the calculation of work only % HRR values above a predetermined threshold (e.g., >40%), reflective of at least moderate exercise. An additional method involves multiplying the mean % HRR above the predetermined threshold by the total duration.

A primary expression of intensity throughout the clinical community is metabolic equivalents (METS). METS is a measure of Intensity or functional capacity. One (1) MET is equivalent to the amount of energy used at rest (oxygen uptake of 3.5 ml/(kg*min)), or the resting VO₂.

1MET=3.5 mL/(kg*min)=VO_{2resting}

METS are linked to heart rate intensity. See, Strath et al. “Evaluation of Heart Rate as a Method for Accessing Moderate Intensity Physical Activity,” Med. & Sci. in Sports & Exerc., 465-470 (2000).

One method for determining METS has been described by Wilkoff, B. L., et al. (“A Mathematical Model of the Cardiac Chronotropic Response to Exercise,” J. Electrophysiol. 3:176-180 (1989)), in which a mathematical model was developed describing the relationship of percentage metabolic equivalents (% METS) to heart rate intensity using the CAEP and Bruce exercise protocols. They found that the relationship was linear, with a slope of approximately 1 (1.06), by the equation:

% METS=1.06*(% HRR)−4.87

Observed METS during exercise can be obtained through the following equation:

$$\% METS = \frac{( {METS}_{observed} - {METS}_{rest} )}{( {METS}_{max} - {METS}_{rest} )} * 100 \% with {METS}_{rest} = 1$$

The value for METSₘₐₓ to be used in the above equation may be obtained as follows:

Predicted METSₘₐₓ=16.6−0.16(age)

This predicted METSₘₐₓ value is an approximation, as it was obtained by a nomogram of sedentary men who participated in the USAir Force School of Aerospace Medicine Protocol, and who did not have a history of CHF. See Morris et al., “Nomogram Based on Metabolic Equivalents and Age for Assessing Aerobic Exercise Capacity in Men,” J. Am. Coll. Cardiol. 22:175-182 (1993). However, if this approximation is used as a best fit method for maximal METS expected for each patient, METS_{observed} can thus be calculated as:

METS_{observed}=(% METS/100)*((16.6−0.16*(age))−1)+1

METS can also be determined by the following method by alternatively solving for % VO₂ reserve. % VO₂ reserve can be calculated by the following equation:

$$\% {VO}_{2} reserve = \frac{( {VO}_{2 observed} - {VO}_{2 rest} )}{( {VO}_{2 max} - {VO}_{2 rest} )} * 100 \% with {VO}_{2 rest} = 1$$

where VO₂ₘₐₓ can be obtained from the non-exercise prediction equation of Jackson et al., “Prediction of functional aerobic capacity without exercise testing,” Med. Sci. Sports & Exerc J. 22:863-870 (1990) by:

VO₂ₘₐₓ=50.513+1.589*(activity scale[0 . . . 7])−0.289*(age)−0.552*(% fat)+5.863*(F=0,M=1).

Or, for those times when % fat may be difficult to obtain, the following equation by Jackson et al. allows for use of Body Mass Index (BMI):

VO₂ₘₐₓ=56.363+1.921*(activity scale[0 . . . 7])−0.381*(age)−0.754*(BMI)+10.987*(F=0,M=1)

In the above two equations for VO₂ₘₐₓ, activity scale can be related to % HRR as a level of activity, % fat or BMI is either calculated as an average over the population or a value to be uploaded to the ICD, and F and M designate female and male, respectively.

When VO₂ₘₐₓ is plugged back into the % VO₂ equation, VO_{2observed} can be obtained (units of mL/[kg*min]). METS_{observed} can be obtained by dividing VO_{2observed} by 3.5.

Another way to determine VO₂ₘₐₓ is by the Astrand single-stage submaximal method, with the following equation:

VO₂ₘₐₓ=VO_{2observed}*[(Age compensated max. HR−K)/(HR_{observed}−K)]

where K=63 for men and 73 for women. (Astrand, P. O., and Rodah, K., Textbook of Work Physiology, 3^{rd} Ed. New York: McGraw-Hill, 1986, p. 318-325 and 340-358.)

Once METS_{observed} has been calculated, it is possible to get the following values:

Relative Oxygen consumption (ml/(kg*min)): METS/3.5

Absolute Oxygen Uptake (L/min): VOₘₐₓ*Weight

Calories (kcal): 1 L O₂=5 kcal: (VO₂ₘₐₓ*duration)/5

Joules: 1 Kcal=4186 J

If the value for METS has a large standard deviation over the above equations, it can be further worked into a descriptive intensity scale (light, moderate, vigorous) as defined by Ainsworth BE et al., Compendium of physical activities: an update of activity codes and MET intensities, Med Sci. Sports Exerc.; 9:S498-S516 (2000)) where these can be defined by:

METS_{60% max cardiorespiratory capacity}=[0.6*(60−0.55*(age)]/3.5 for men, and

METS_{60% max cardiorespiratory capacity}=[0.6*(48−0.37*(age)]/3.5 for women

with 60% max cardiorespiratory capacity (MCC) considered vigorous. Therefore, light intensity would be, for example, between 20-40%, and moderate activity would be, for example, between 40-60%.

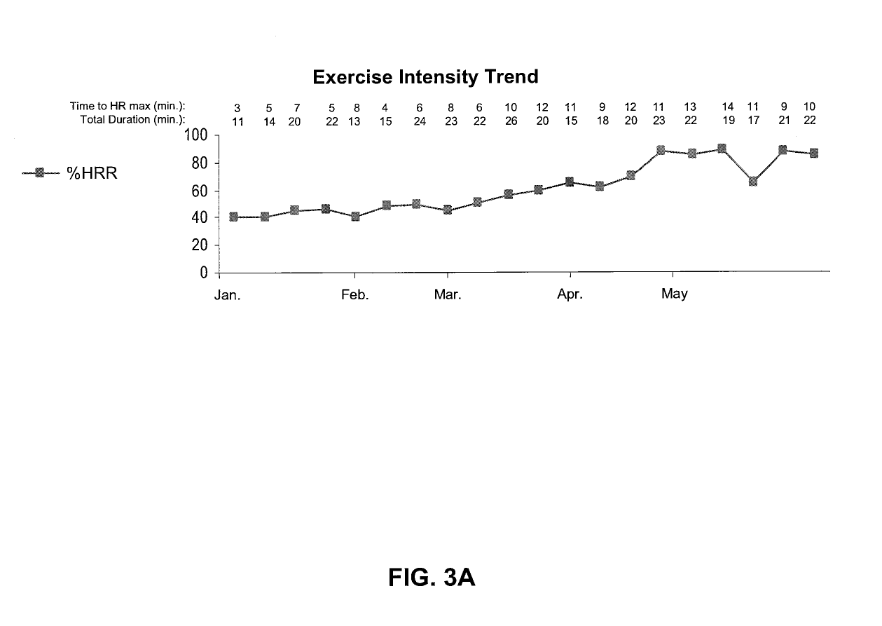

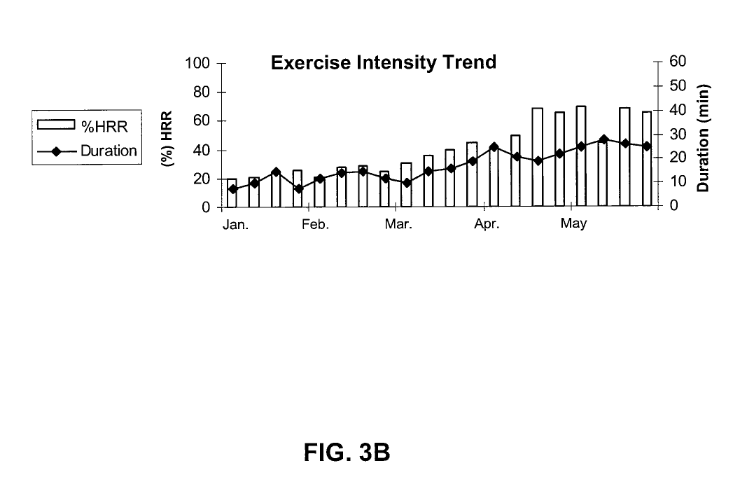

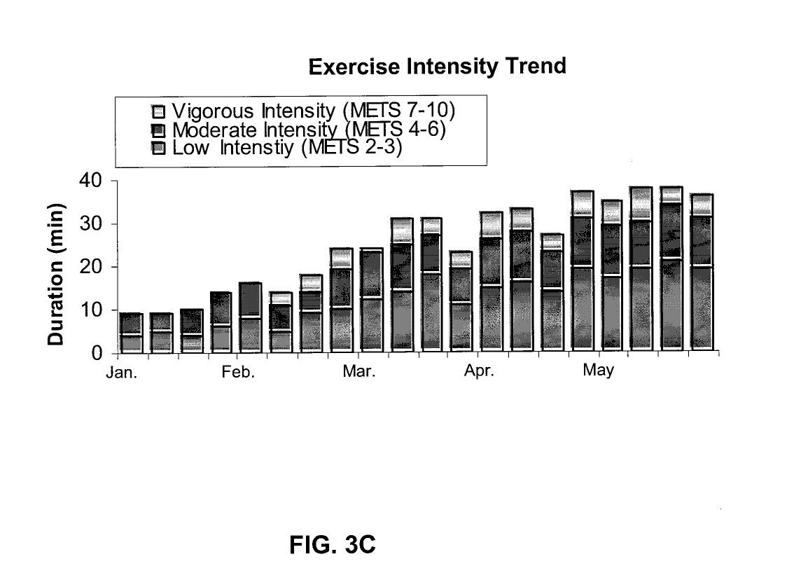

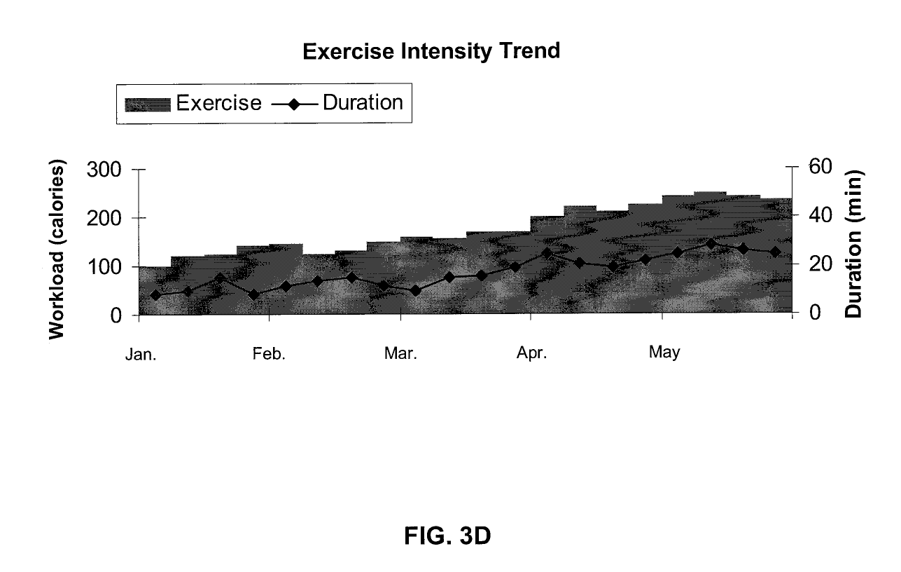

FIGS. 3A to 3D illustrate how exercise data, such as exercise intensities may be displayed. The data illustrated in these Figures are prophetic. In FIG. 3A, measured heart rate intensity (% HRR) data is displayed as a function of time in the graph. The table above the chart illustrates the corresponding time to HRₘₐₓ and the total duration of HRₘₐₓ. In FIG. 3B, measured heart rate intensity and total duration of HRₘₐₓ are illustrated on one graph. In FIG. 3C, measured exercise intensity in the units of METS is illustrated, with corresponding amounts of vigorous, moderate, and low intensities, and the duration of each amount. In FIG. 3D, measured workload is illustrated, with corresponding duration of the workload. Each data point illustrated on the table and graphs of FIGS. 3A-3D represent an average over one week.

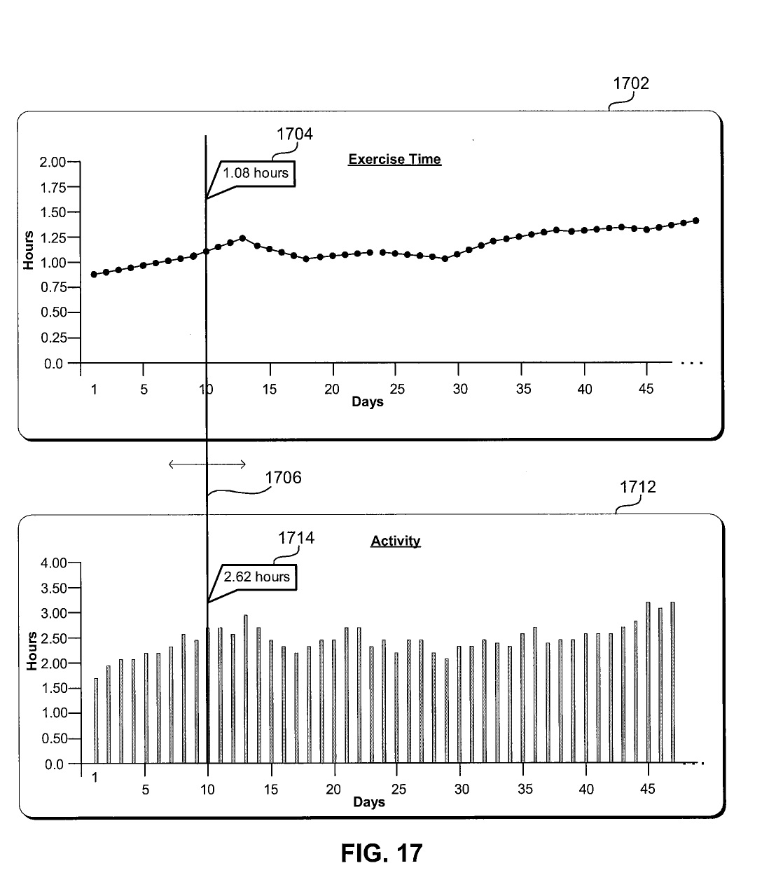

As is illustrated from FIGS. 3A-3D, embodiments of the invention also encompass determining the time period associated with exercise intensities. For example, the time to and duration of HRₘₐₓ and workload can be determined. FIG. 17, described below, illustrates alternative embodiments for displaying exercise data, as well as activity data.

The above-described method 200 for determining the maximum observed heart rate of a patient during exercise may be implemented by hardware, software, or firmware of a pacing system, such as the ICD described earlier with reference to FIGS. 1A and 1B, with particular reference to HRₘₐₓ detector 62.

In another embodiment, a method for determining exercise diagnostics, such as workload, heart rate intensity, percent oxygen consumption (% VO₂) reserve, metabolic equivalents (METS), percentage METS, and absolute oxygen uptake may be obtained without obtaining HRₘₐₓ. This method includes monitoring a changing heart rate of a patient and producing heart measurements, monitoring activity level, and determining an exercise diagnostic, such as workload of the patient using at least one heart rate measurement when the activity level exceeds an activity threshold.

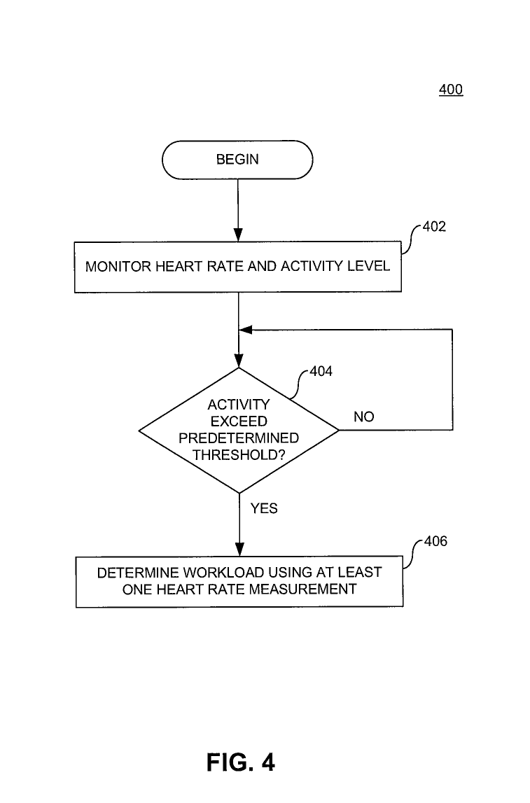

A method 400 of determining workload of a patient during exercise is illustrated in FIG. 4. According to an embodiment, the method 400 begins at step 402, in which the heart rate and activity level of the patient is monitored. The heart rate and activity level of the patient may be continuously monitored during the method 400.

As discussed above in conjunction with the method for determining HRₘₐₓ, the patient's heart rate and activity level may be determined by any suitable method, and heart rate measurements can be generated based upon the monitored heart rate. In embodiments, the heart rate measurements include heart rate intensity.

In step 404, the measured activity level is compared with a predetermined activity threshold to determine whether the activity level exceeds the threshold. As discussed above, the predetermined activity threshold can be a value that corresponds to a certain level of exercise and can be tailored for a specific patient's condition.

The activity level can be compared with an activity threshold at various time intervals to determine whether the activity level exceeds the predetermined threshold for a predetermined period of time. The time interval or frequency of comparing the activity level with the activity threshold is not critical to embodiments of the invention. In an embodiment, the activity level is monitored and compared with the activity threshold at time intervals of 30 seconds.

If it is determined in step 404 that the patient activity level exceeds a predetermined activity threshold, then step 406 is performed. As discussed above in conjunction with determining HRₘₐₓ, step 406 can be performed when the patient activity level exceeds the predetermined activity threshold for at least a predetermined period of time. This predetermined period of time may correlate to the amount of time for the heart to react to the exercise by the patient. Illustratively, the predetermined period of time may be 10 seconds to five minutes, preferably about two to three minutes, more preferably about two minutes.

In step 406, workload of the patient is determined using at least one heart rate measurement. Preferably, a heart rate measurement that is used to determine work of the patient during the exercise is heart rate intensity.

Specifically, workload of a patient during exercise can be determined by the summation of intensities over time over the full time of exercise (i.e., for the entire period that the activity level exceeds the predetermined threshold), where intensities are calculated from the previous equations discussed to obtain VO_{2observed}. Alternately, as discussed above, workload may be described as a unitless index by multiplying intensities such as % HRR or % VO₂ reserve and time. Illustratively, after the activity level exceeds an activity threshold, work values can be calculated (Intensity*Duration) for each data-point until the cessation of exercise (i.e., when the activity level no longer exceeds the predetermined threshold). The determination of work of the patient during the exercise can also be represented by the following formula:

ΣIntensity(x)*(Time(x)−Time(x−1))

where x=0:n.

Based on the workload value obtained above, other exercise diagnostics, such as heart rate intensity, percent oxygen consumption (% VO₂) reserve, metabolic equivalents (METS), percentage METS, and absolute oxygen uptake may be obtained. For example, heart rate intensity may be found by dividing the work by the total time of exercise.

The above-described method 400 for determining workload of a patient during exercise may be implemented by hardware, software, or firmware of a pacing system, such as the ICD described earlier with reference to FIGS. 1A and 1B, with particular reference to work detector 64.

Heart rate recovery (HRR) involves analyzing how the heart recovers from a maximum rate during exercise. The heart rate recovery value may not change in a matter of days, but possibly in a matter of weeks. Obtaining the heart rate recovery value only during episodes of peak exercise, as opposed to any low-level exercise, may provide a more accurate reflection of cardiac health through heart rate recovery.

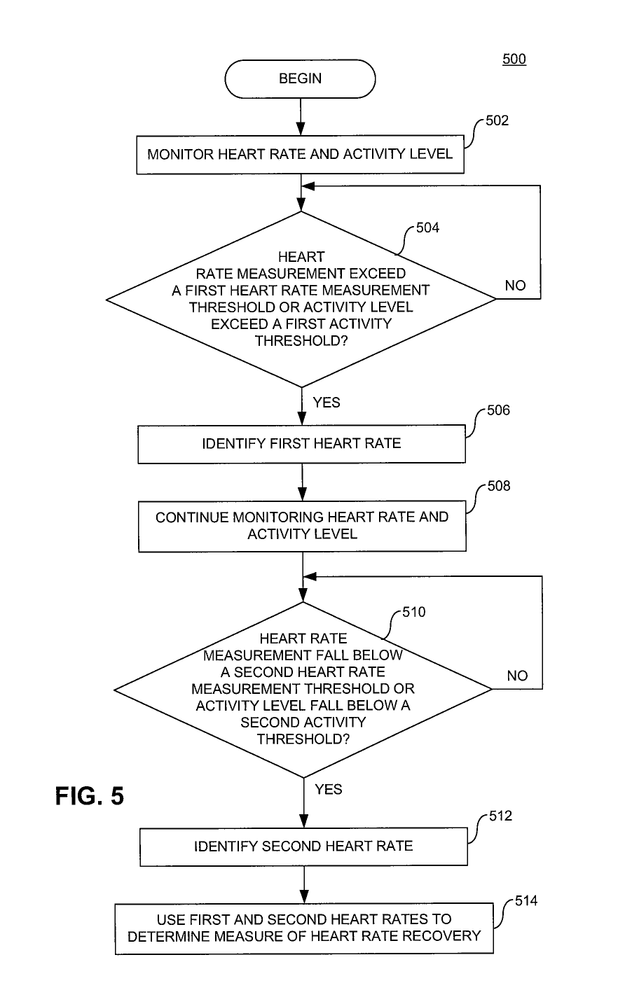

A method 500 of determining a measure of heart rate recovery is illustrated in FIG. 5. According to an embodiment, the method 500 begins at step 502, in which the heart rate and activity level of the patient are monitored. The heart rate and activity level of the patient may be continuously monitored during the method 500. The heart rate and activity level can be monitored by any suitable method, including those discussed above.

Heart rate measurements can be produced based upon the monitored heart rate. As discussed above, such heart rate measurements include but are not limited to heart rate and heart rate intensity.

In step 504, a heart rate measurement is compared with a first heart rate measurement threshold, and an activity level is compared with a first activity threshold. The first heart rate measurement threshold and first activity threshold may be indicative of exercise, preferably vigorous or peak exercise.

In step 504, if a heart rate measurement exceeds a first heart rate measurement threshold, and/or an activity level exceeds a first activity threshold, then the method proceeds to step 506. In step 506, the heart rate is identified as a first heart rate. That is, the heart rate taken at the time (1) a heart rate measurement exceeded a first heart rate measurement threshold and/or (2) the activity level exceeded the first activity threshold is used as a first heart rate value for further computations.

In one embodiment, in step 504, the first heart rate is identified when at least one heart rate measurement exceeds the first heart rate measurement threshold. In another embodiment, the first heart rate is identified when at least one heart rate measurement exceeds the first heart rate measurement threshold for a predetermined period of time. In other embodiments, the first heart rate can be identified when an average value of heart measurements (taken over a predetermined time period, such as, for example, one minute) exceeds the first heart rate measurement threshold.

In another embodiment, the first heart rate can be identified when the activity level exceeds the first activity threshold. In yet another embodiment, the first heart rate can be identified when the activity level exceeds the first activity threshold for a predetermined period of time. In still yet another embodiment, the first heart rate can be identified when, for the predetermined period of time, an average activity level exceeds the first activity threshold.

Preferably, the first heart rate is identified when both the activity level exceeds a first activity threshold and a heart rate measurement exceeds a first heart rate measurement threshold.

Even more preferably, the first heart rate is identified when the mean activity level value exceeds a first activity threshold for a predetermined period of time, and a mean heart rate measurement value, such as heart rate intensity, exceeds a first heart rate measurement threshold for a predetermined period of time.

In accordance with some embodiments, the first heart rate is identified only during peak exercise, only after a stringent set of conditions have been met. These conditions can include certain levels of heart rate intensity, activity level and duration of time. This first heart rate may be referred to as a peak exercise heart rate.

Illustratively, a peak exercise heart rate can be identified when the mean activity level exceeds a first activity threshold and the heart rate intensity exceeds a heart rate intensity threshold, such as, e.g., 80%, for a period of time of at least about five minutes.

As illustrated by step 508, heart rate and activity level continue to be monitored. It should be understood that the identified first heart rate can be overwritten by a subsequent heart rate (including a slower heart rate), provided that the first heart rate criteria described above are still met.

Heart rate and activity level also continue to be monitored, as illustrated by step 508, for determining the next parameter used to determine heart rate recovery, a second heart rate. The second heart rate is the heart rate after a slow-down in exercise, and is compared with the first heart rate to determine a measure of heart rate recovery. In accordance with some embodiments, heart rate measurements (such as, for example heart rate) continued to be produced.

In step 510, a heart rate measurement is compared with a second heart rate measurement threshold, and an activity level is compared with a second activity threshold. The second heart rate measurement threshold and second activity threshold can be indicative of a slowing down or cessation of exercise.

If a heart rate measurement falls below a second heart rate measurement threshold, and/or an activity level falls below a second activity threshold, then in step 512 the monitored heart rate is identified as a second heart rate.

In one embodiment, the second heart rate is identified when the activity level falls below the second activity threshold for a predetermined period of time. Preferably, the second heart rate is identified when a mean activity level falls below the second activity threshold for a predetermined period of time. The comparison can also be done based on an average activity level over a predetermined period of time.

In another embodiment, the second heart rate is identified when a heart rate measurement falls below a second heart rate measurement threshold for a predetermined period of time. For example, if a heart rate measurement (e.g. heart rate) falls below a predetermined threshold and/or the mean activity level falls below a predetermined activity threshold, then the heart rate and activity levels can be recorded for a predetermined period of time, such as, for example one, two, or three minutes.

After the predetermined period of time, if a heart rate measurement is less than the heart rate measurement prior to the predetermined period of time, and the activity level is less than a third activity threshold (which can be the same as or lower than the second activity threshold), then a second heart rate is identified. Preferably, the slowest heart rate measured during the predetermined period of time is identified as the second heart rate.

In step 514, once a first heart rate and a second heart rate are identified, the first and second heart rates are used to determine a measure of heart rate recovery. For example, the second heart rate is subtracted from the first heart rate to obtain a heart rate difference. The difference is a heart rate recovery value.

It should be understood that additional second heart rate values can be identified after the first heart rate and compared to the first heart rate to determine a measure of heart rate recovery. Accordingly, the term “second heart rate” is intended to encompass one or more heart rates that meet the above-described criteria for identification of the second heart rate. In other words, the second heart rate may be several heart rates over consecutive periods of time (e.g. minutes).

Illustratively, heart rates measured at discrete times after the identified first heart rate and that meet the second heart rate identification criteria can be compared with the first heart rate to determine a measure of heart rate recovery. For example, the difference between the first heart rate and each of the second heart rates can provide a measure of heart rate recovery. Also, a listing of the first heart rate and heart rates meeting the second heart rate criteria as they decrease over time can also be a measure of heart rate recovery.

Embodiments of the invention also encompass identifying the first heart rate at the time the criteria for identifying the second heart rate is met. For example, if a heart rate measurement exceeds a first heart rate measurement threshold or an activity level exceeds a first activity threshold, and subsequently a heart rate measurement falls below a second heart rate measurement or the activity level falls below a second activity threshold, a first heart rate can be identified at or near the inflection point between meeting the first and second heart rate identification criteria.

The second heart rate then can be identified as one or more heart rates measured subsequent to the identified first heart rate. For example, provided that the measured heart rates meet the second heart rate identification criteria, a second heart rate can be identified one minute, two minutes, and/or three minutes following the first heart rate. The difference between the first heart rate and the second heart rate at one, two, and/or three minutes post-first heart rate identification provides values that determine a measure of heart rate recovery.

To illustrate, a patient exercises (e.g. runs) for five minutes, and then stops running and sits down for three minutes. Provided that the patient met the first and second heart rate identification criteria described above, the first heart rate would be identified at the five minute mark, and the second heart rates would be identified at the six, seven, and eight minute mark. The first heart rate would be compared with each of the second heart rates at the six, seven, and eight minute mark to determine a measure of heart rate recovery.

In accordance with a specific embodiment, in the method 500 for determining a measure of heart rate recovery, heart rate measurements are filtered to remove noise and premature heart beats such as arrhythmias, PACs, and PVCs.

The above-described method 500 for determining the measure of heart rate recovery of a patient may be implemented in software, or firmware of a pacing system, such as the ICD described earlier with reference to FIGS. 1A and 1B, with particular reference to HR Recovery Detector 66.

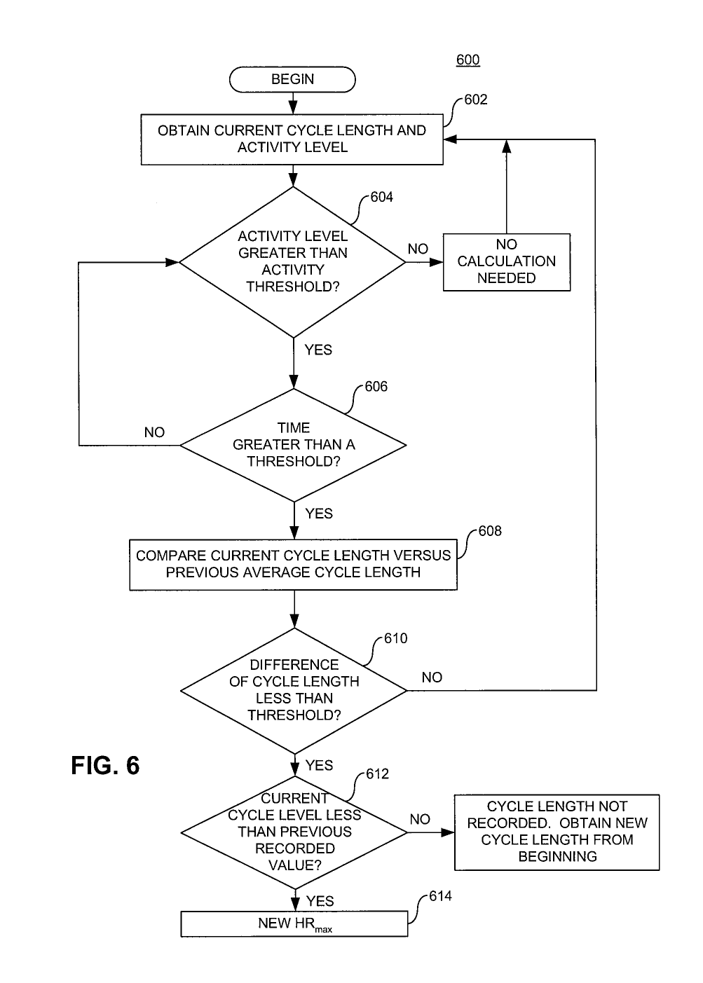

A method for determining a maximum observed heart rate (HRₘₐₓ) of a patient during exercise is illustrated in FIG. 6.

To calculate HRₘₐₓ for exercise conditioning, it is preferred that the patient has maintained a certain level of activity for a certain period of time. Thus, in the illustrative method, the maximum observed heart rate is not calculated unless the activity level is above a threshold activity for a certain period of time.

In method 600, the current cycle length (inverse of heart rate) and activity level are obtained as illustrated in step 602. It should be understood that the cycle length and activity level can be continuously or periodically monitored.

In step 604, the activity level measured is compared with an activity threshold. If the activity is less than an activity threshold, then the method returns to step 602. In this manner, steps 602 and 604 result in a continuous (or, optionally, periodic) monitoring of activity level.

If the measured activity level is greater than the activity threshold, then in step 606 the elapsed time (i.e., the period during which the activity level is greater than the activity threshold) is compared with a time threshold. The time threshold can be, for example, 2-3 minutes. Once this comparison indicates that sufficient time has elapsed, then step 608 is performed. Thus, before step 608 is performed, there has been a sufficient activity level for a sufficient period of time to indicate actual exercise by the patient.

In step 608, the current cycle length is compared with the previous cycle lengths, preferably a previous average cycle length. In step 610, if the difference of cycle length is too large (e.g., greater than or equal to about 100 milliseconds), this may indicate noise, PACs, PVCs or arrhythmias, and will not be identified as the HRₘₐₓ. If this occurs, as illustrated in step 610, the method returns to step 602 to obtain a new, current cycle length and activity.

In step 610, if the difference of cycle length is less than a threshold (indicating that the current cycle length is not due to noise or a premature heart beat), then in step 612, the current cycle length is compared with the previous cycle length. If the current recorded cycle length is not less than the previously recorded value, then the current cycle length is not identified as the HRₘₐₓ, and step 602 is repeated. However, if the current cycle length is less than the previous recorded value, then in step 614 the current cycle length is identified and stored as the new HRₘₐₓ.

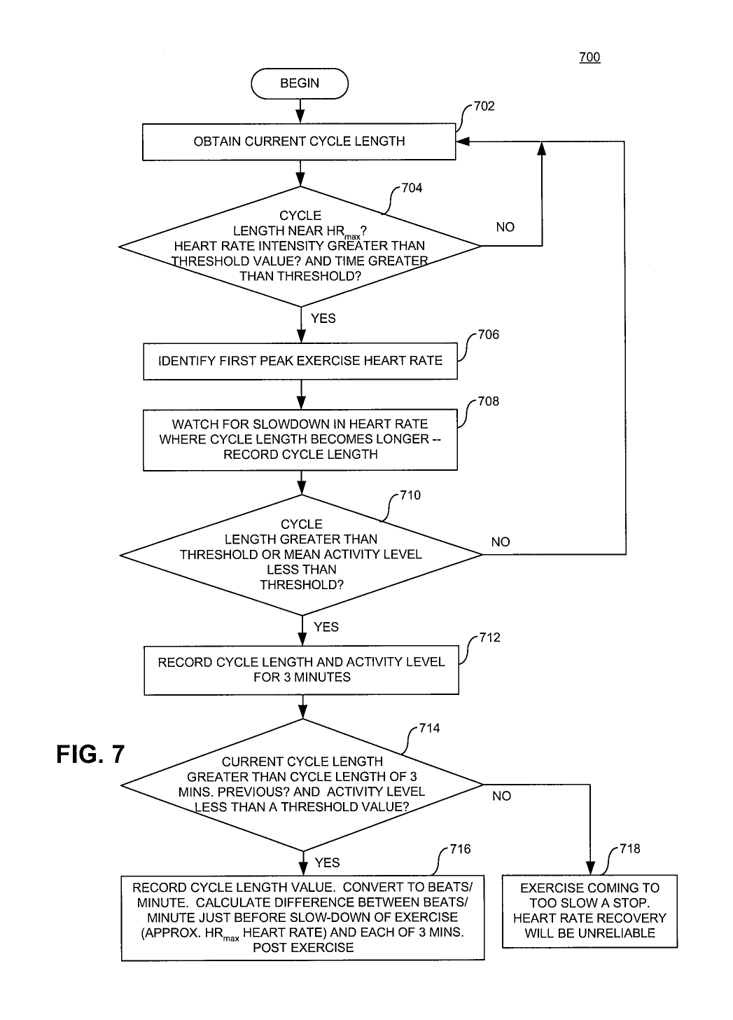

A method for determining heart rate recovery of a patient is illustrated in FIG. 7.

In accordance with the illustrated method 700, the first heart rate, a peak exercise heart rate is obtained by analyzing cycle lengths only when the qualifications for HRₘₐₓ have been met.

In step 702, the current cycle length is obtained. In step 704, if the cycle length is near HRₘₐₓ, the value of the heart rate intensity is determined. If this value is greater than a predetermined threshold such as, e.g., 65%, and if the length of time is greater than a predetermined threshold, such as, e.g., 5 minutes, then in step 706 the cycle length is recorded as the first, peak exercise heart rate. Otherwise, the heart rate intensity for each cycle length continues to be determined.

The cycle length recorded in step 706 is continuously or periodically recorded, and may be overwritten by slower rates. However, if a noticeable slowdown occurs, in step 708 a new buffer collects the recorded cycle length. In step 710, if the current cycle length is greater than a predetermined threshold, such as, e.g., 20 milliseconds, or the mean activity level is less than a predetermined threshold, each indicative of a drop in activity, then in step 712 cycle length values are continuously recorded for three minutes.

In step 714, if after three minutes, the cycle length is greater than the cycle length measured three minutes previously, and the activity level is less than a threshold value, then in step 716 the largest cycle length for each of the three minutes is recorded as the second set of heart rate recovery values. If these criteria in step 714 are not met, then in step 718 a second heart rate is not recorded, as the exercise is coming too slowly to a stop.

Once the cessation of exercise has been determined, in step 716 both the first, peak exercise heart rate and the three heart rate recovery value cycle lengths are converted to beats per minute and subtracted from each other. The values obtained are the times of heart rate recovery.

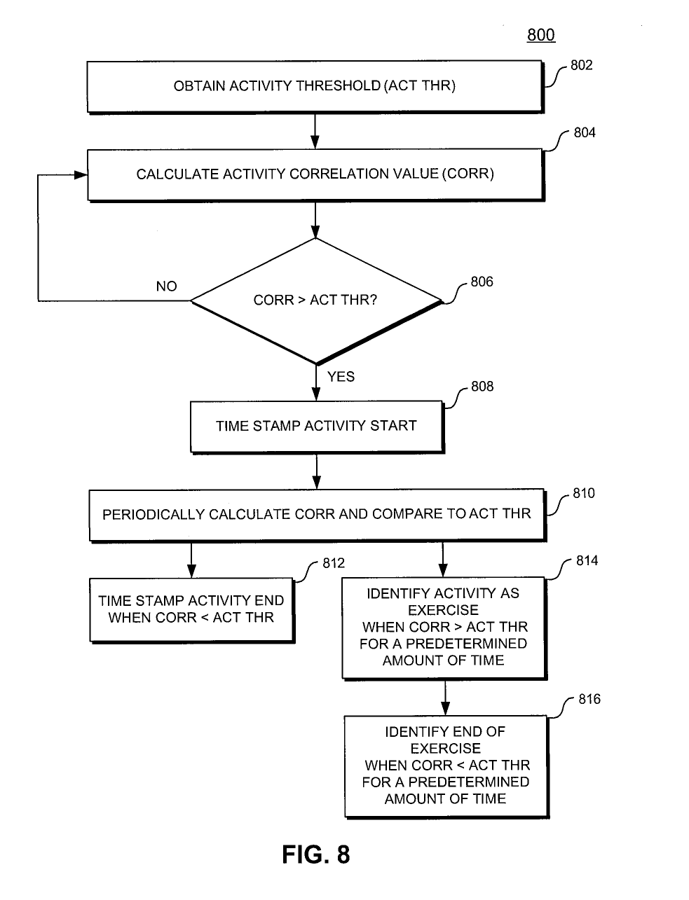

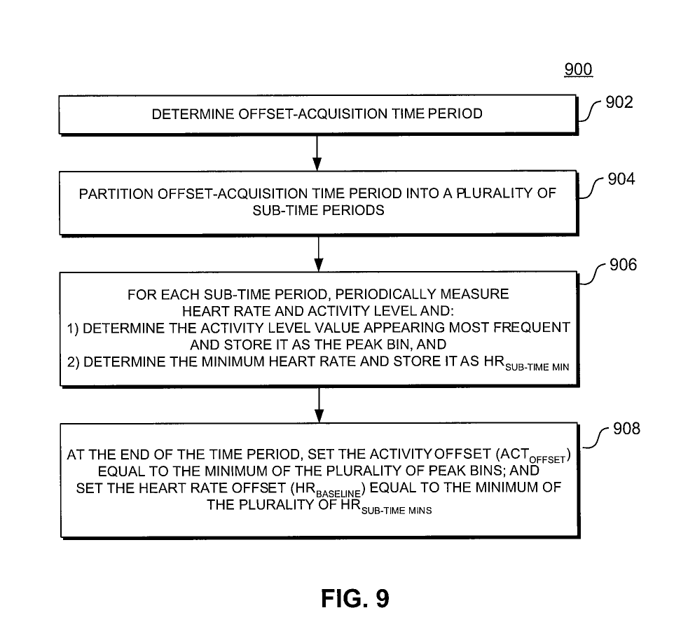

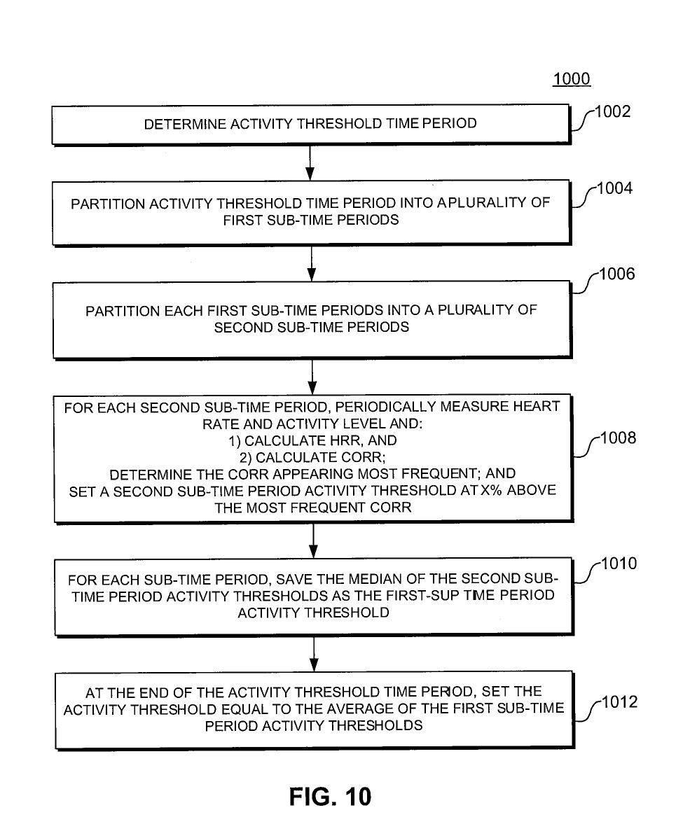

A method 800 of determining the duration of patient activity and patient exercise is illustrated in FIG. 8. According to an embodiment, the method 800 begins at step 802, in which a patient-specific activity threshold is obtained, e.g., calculated based on historical heart-rate data and historical activity-level data, which is collected over a period of time, and predetermined patient-specific offset parameters. Details related to the predetermination of patient-specific offset parameters and the determination of the patient-specific activity threshold are provided below with reference to FIGS. 9 and 10, respectively.

Continuing with FIG. 8, in step 804, current heart-rate and current activity level measurements are used to calculate a current activity correlation value (CORR_{current}) using the following equation:

CORR_{current}=HRR×(ACT_{current}−ACT_{offset}),

where HRR (heart rate reserve) can be obtained as described below with reference to FIG. 10, step 1008; ACT_{current} is the activity measurement output by an activity sensor; and ACT_{offset} is obtained as described below with reference to FIG. 9, step 908.

In a preferred embodiment, the heart rate and activity level measurements are taken periodically, for example, every “x” seconds or “n” heart beats where “x” and “n” are programmed and may be small quantities, such as 5 seconds and 3 heart beats, where resolution increases accuracy of detection. Periodic sampling is preferred over continuous measurements in order to conserve memory. In step 806, the current activity correlation value (CORR_{current}) is compared to the activity threshold (ACT THR). If the current correlation value does not exceed the activity threshold, the process returns to the step 804, where another current activity correlation value is calculated using the next periodic current heart-rate and activity level measurements, and the comparison process repeats. If at any time a current activity correlation value exceeds the activity threshold a beginning-of-activity time-stamp is created at step 808.

At step 810, the method continues to calculate current activity correlation values and compares each to the activity threshold. At step 812, if an activity correlation value fails to exceed the threshold, an end-of-activity time-stamp is created. As explained further below, the start and end time stamps provide a measure of the duration of activity. Numerous activity durations over a period of time may be used to report activity related quantities. For example, a total daily activity duration can be created by summing all the periods of activity that occurred over the course of a day.

At step 814, if current correlation values continue to exceed the activity threshold value for a predetermined amount of time, the activity is considered to have been sustained long enough to qualify as exercise. For example, assuming the predetermined amount of time is five minutes, 10 consecutive activity correlation values (calculated every 30 seconds) exceeding the activity threshold value would qualify the activity as exercise. The predetermined amount of time is programmable. As explained further below, if activity is identified as exercise, the predetermined amount of time associated with step 814 may be subsequently included in an exercise duration calculation.

At step 816, once an exercise state has been identified, the current correlation values continue to be monitored and if they fall below the activity threshold value for a predetermined amount of time, the exercise period is considered to have ended. This predetermined amount of time, which is also programmable, serves to distinguish between a pause in exercise and a stop of exercise. For example, for a predetermined amount of time equal to one minute, at least six consecutive current correlation (CORR_{current}) values (calculated every 10 seconds) would have to fail to exceed the activity threshold to signify an end of exercise. Less than six current correlation values failing to exceed the activity threshold would be considered a pause in exercise. As explained further below, if an end of exercise is identified, the predetermined amount of time associated with step 816 may be subsequently included in an exercise duration calculation.

With reference to FIG. 9, a method 900 of determining the patient-specific offset parameters (ACT_{offset} and HR_{baseline}) is illustrated. The data collection of offset parameters are typically initiated upon implant of the device, and may be periodically updated to account for changes in the patient's condition and sensitivity of the device sensors. The method involves the collection and analysis of patient data over a period of time, referred to as the “offset-acquisition time period.”

According to an embodiment, the method 900 begins at step 902, in which the offset-acquisition time period is determined. This time period may be programmed into the device by the physician and is long enough to allow for the collection and analysis of a quantity of patient data sufficient to avoid the potential for inaccurate parameter offset calculations due to possible noise and atypical patient activity. In one configuration, the offset-acquisition time period is 24 hours. In step 904 the offset-acquisition time period is partitioned into sub-time periods, for example, 1 hour time periods in the case of a 24 hour offset-acquisition time period. Though the use of sub-time periods is not necessary, as will be apparent from the continuing explanation of the method 900, sub-time periods conserve memory space.

At step 906, for each sub-time period, heart rate and activity level are periodically measured, for example every 10 seconds. The patient's heart rate may be determined by any suitable method. Many variations on how to determine heart rate are known to those of ordinary skill in the art, and any of these of reasonable accuracy may be used. Heart rate can be determined by measurement of an R-R interval cycle length (or P-P), which is the inverse of heart rate. As used herein, the heart rate (in beats per minute) can be seen as the inverse to cycle length, determined by 60,000 divided by the cycle length (in milliseconds).

The activity level of the patient may also be determined by any suitable method. For example, the activity level may be determined by an accelerometer, piezoelectric crystal, minute ventilation, or a derivative thereof, such as the sensor indicated rate. In one embodiment, activity level is determined using physiologic sensor 108. In this embodiment, sensor 108 is an accelerometer, a piezoelectric crystal or an impedance sensor, but is not limited thereto.

Continuing with FIG. 9, step 906, for each sub-time period, in an exemplary type of activity data analysis, a histogram of activity level data over the sub-time period is created. At the end of the sub-time period, the activity level value appearing most frequent within the 1 hour histogram is saved as the “peak bin.” In addition, at the end of each sub-time period, in an exemplary type of heart-rate data analysis, a minimum heart rate measured in the sub-time period is stored as HR_{sub-time min}. The HR_{sub-time min} is not necessarily the lowest heart rate measured but instead may be a heart rate that is close to the lowest measured rate in order to account for possible noise.

At step 908, at the end of the time period, the activity offset parameter (ACT_{offset}) is determined to be the minimum of the plurality of activity-level “peak bins.” The heart rate offset (HR_{baseline}) is determined to be the minimum of the HR_{sub-time min}'s measured during the period of time.

With reference to FIG. 10, a method 1000 of determining the patient specific activity threshold (ACT THR) of step 802 of the method of FIG. 8 is illustrated. The activity threshold is typically determined upon implant of the device, and may be periodically updated to account for changes in the patient's condition and sensitivity of the device sensors. The method involves the collection and analysis of patient data over a period of time, referred to as the “activity threshold time period.”

According to an embodiment, the method 1000 begins at step 1002, in which the activity threshold time period is determined. This time period may be programmed into the device by the physician and is long enough to allow for the collection and analysis of a quantity of patient data sufficient to avoid the potential for inaccurate activity threshold calculations due to possible noise and atypical patient activity. In one configuration, the activity threshold time period is 7 days. In step 1004 the activity threshold time period is partitioned into first sub-time periods, for example 1 day time periods, in the case of a 7 day activity threshold time period. In step 1006 the first sub-time periods are partitioned into second sub-time periods, for example, 1 hour time periods in the case of 1 day first sub-time periods. As described above with reference to the determination of patient-specific offset parameters, the use of sub-time periods is not necessary, but is preferred in order to conserve memory space.

At step 1008, for each second sub-time time period, heart rate and activity level are periodically measured, for example every 10 seconds. The heart rate measurements are used, in turn, to calculate a plurality of heart-rate reserve (HRR) values using the following equation:

$$HRR = \frac{HR - {HR}_{baseline}}{Age Compensated Maximum HR - {HR}_{baseline}} \times 100$$

Heart-rate reserve is used in the method instead of heart rate in order to utilize a heart rate measurement that is normalized across the patient population. Such normalization accounts for the fact that the same heart rate may correspond to different activity states for different people. For example, a heart rate of 90 beats per minute may correspond to sitting for one person and walking for another person. In the equation indicated above, HR (heart rate) may be obtained by any suitable method; HR_{baseline} may be obtained as described with reference to FIG. 9, and age compensated maximum heart rate can be calculated by the formula: (220-age).

While the above HRR equation is expressed in terms of HR parameters, actual implementation of the above HRR equation may involve the use of heart rate interval (HRI) calculations. In terms of HRI, the equation for HRR becomes:

$$HRR = \frac{\frac{{HRI}_{min}}{HRI} \times ( {HRI}_{baseline} - HRI )}{{HRI}_{baseline} - {HRI}_{min}} \times 100$$

In the equation indicated above, HRI (heart rate interval) may be obtained by any suitable method; HRIₘᵢₙ may be obtained using the equation: 60,000/(220−age) and HRI_{baseline} may be obtained by converting the HR_{baseline} measurement described with reference to FIG. 9 into a heart rate interval measurement.

In order to reduce rounding errors when processing the above equation, it is desirable to perform all multiplication functions first in order to make the numerator larger than the denominator. Considering the range of HRI in milliseconds, a 2 byte by 2 byte multiplier may be required to perform the multiplication operations. In addition, there are several multiplication and division operations in the HRR equation, which may impact processor duty cycle.

In an alternate embodiment, processing efficiency may be enhanced by obtaining an approximated heart-rate reserve using the following equation:

HRRₐₚₚᵣₒₓ=HR−HR_{baseline}

which in terms of HRI translates to:

HRRₐₚₚᵣₒₓ=(60,000/HRI)−(60,000/HRI_{baseline})

This calculation reduces the number of arithmetic operations and may be completed using a 1 byte by 1 byte multiplier; thereby reducing the processor duty cycle.

Continuing with step 1008, the activity level measurements are used, in turn to calculate a plurality of activity correlation values using the following equation:

CORR_{sub-time}=HRR×(ACT_{sub-time}−ACT_{offset})

where ACT_{sub-time} is the activity measurement provided by the activity sensor during the sub-time period; and ACT_{offset} is obtained as described above with reference to FIG. 9, step 908. Note that HRRₐₚₚᵣₒₓ may be used in place of HRR in the CORR_{sub-time} calculation.

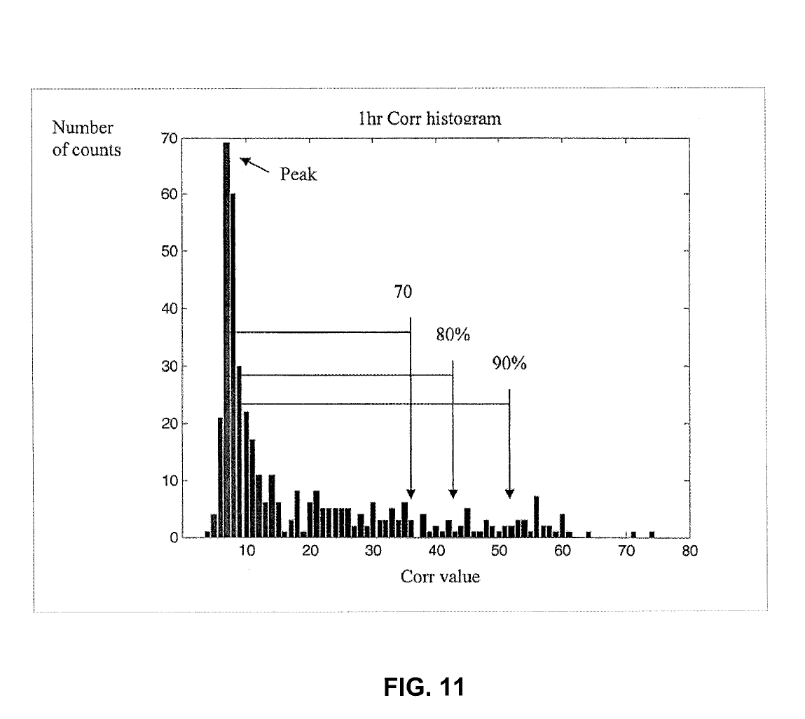

The sub-time correlation values are analyzed using, for example, a histogram analysis similar to that described above with respect to FIG. 9, step 906, to identify the sub-time correlation value appearing most frequent within the second sub-time period. A second sub-time activity threshold is set at X % above the most frequent correlation value, where X is programmable and may be for example, 70, 80 or 90. Setting the second sub-time activity threshold as a percentage above the most frequent correlation value provides for the filtering of noise and allows for a deterministic approach towards a threshold that encompasses a level of activity. An example of a 1 hour histogram is shown in FIG. 11.

Continuing with FIG. 10, in step 1010 after identifying the plurality of second sub-time period activity thresholds, e.g., twenty-four, 1 hour thresholds in the case of a 1 day first sub-time period, the median of the plurality of second sub-time period activity thresholds is identified as the activity threshold for that first sub-time period. While other statistical measurements may be used, the median value is chosen for robustness of the threshold, which will not be changed by several hours of atypical activities. The process is repeated for each first sub-time period to identify a plurality of first sub-time period activity thresholds. Thus in the case of a 7 day activity threshold time period, seven, 1 day activity thresholds would be determined. In step 1012, at the end of the total activity threshold time period, e.g., 7 days, the activity threshold (ACT THR) is set equal to the average of the plurality of first sub-time period, e.g., 1 day, activity thresholds. While other statistical measures may be used, an average is chosen to account for weekday and weekend difference of activity patterns.

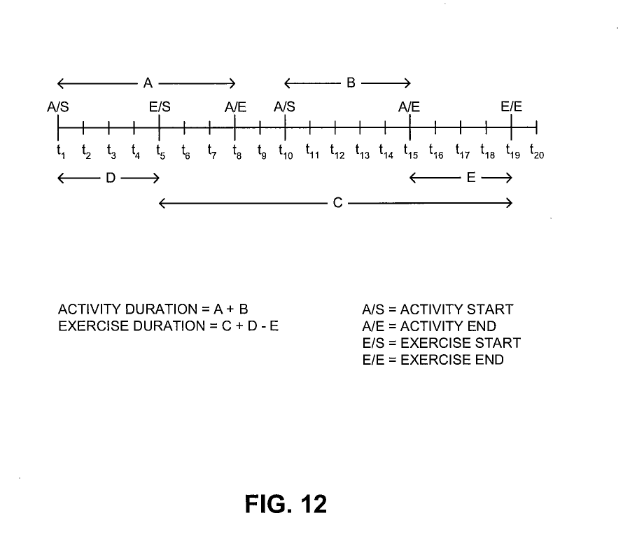

With reference to FIG. 12, various activity and exercise states identified over a period of time in accordance with the method 800 described above with reference to FIG. 8 is presented in a time line to aid in describing possible activity duration and exercise duration calculations. At time t₁, CORR_{current} exceeds ACT THR and a start of activity (A/S) is identified. For each of times t₂ through t₇, CORR exceeds ACT THR. The time t₁ through t₅ corresponds to the predetermined “exercise” time used to identify exercise; accordingly, a start of exercise (E/S) is identified at time t₅.

At time t₈ CORR did not exceed ACT THR and an end of activity (A/E) is identified. At time t₁₀ CORR again exceeds ACT THR and a start of activity (A/S) is identified. Because the time between t₈ (when CORR first failed to exceed ACT THR) and t₁₀ (when CORR again exceeded ACT THR) is less that the predetermined time used to identify the end of exercise (an “exercise hysteresis”), this period of time is considered to be a pause in exercise and there is no end of exercise identification. At time t₁₅, CORR failed to exceed ACT THR and an end of activity (A/E) is identified. At each of times t₁₆ through t₁₉, CORR fails to exceed ACT THR. Because the time between t₁₅ and t₁₉ corresponds to the predetermined time used to identify the end of exercise, an end of exercise (E/E) is identified at time t₁₉.

From the start and end identifications included in this exemplary time line, activity duration would be calculated as A+B where A is the duration of the first activity period and B is the duration of the second activity period. Exercise duration would be calculated as C+D−E, where C is the difference between the start of exercise (E/S) and the end of exercise (E/E), D is the predetermined time used to identify the start of exercise and E is the predetermined time used to identify the end of exercise.

Graphs of heart rate reserve data and activity sensor data as a function of time corresponding to patient activity on a Stairmaster, correlation values as a function of time corresponding to the same patient activity on a Stairmaster, heart rate reserve data and activity sensor data as a function of time corresponding to patient activity on a treadmill, and correlation values as a function of time corresponding to the same patient activity on a treadmill, are provided in U.S. patent application Ser. No. 11/405,129, filed Apr. 13, 2006, entitled “Methods and Devices For Determining Exercise Compliance Diagnostics”, which was incorporated herein by reference above.

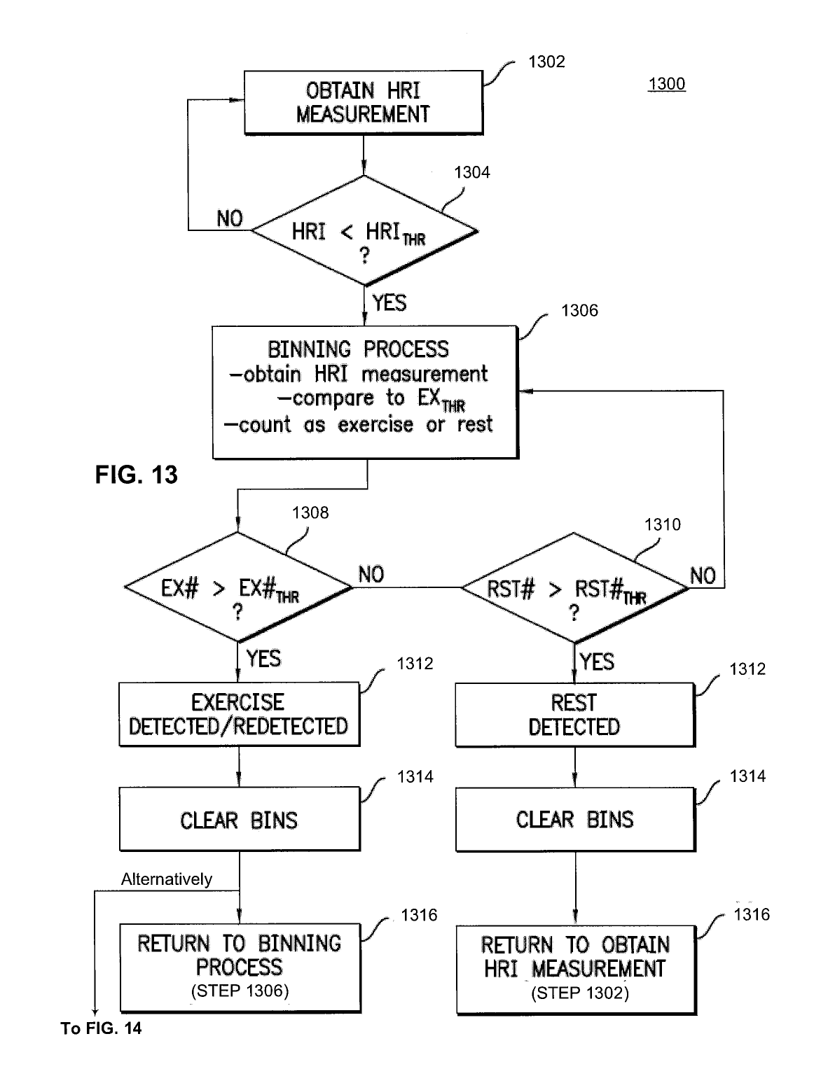

The flow chart of FIG. 13 will now be used to describe a method 1300 for detecting exercise and/or rest of a patient based on heart rate data. Referring to FIG. 13, at step 1302 a heart-rate-interval (HRI) measurement of the patient is obtained. In one embodiment, the HRI measurement corresponds to a cardiac event interval, which is defined by at least two cardiac events. For example, a cardiac event interval may be the interval between consecutive ventricular events, e.g., R waves, or consecutive atrial events, e.g., P waves, and the HRI measurement corresponding to the cardiac event interval may simply be the duration of interval. Alternatively, the HRI measurement may be expressed in terms of beats-per-minute, in which case the HRI measurement would be 60,000 divided by the time interval in milliseconds. The HRI measurements may be obtained by any suitable method. Many variations on how to determine heart rate are known to those of ordinary skill in the art, and any of these of reasonable accuracy may be used.

In another arrangement, the HRI measurement for a particular cardiac event interval may be based on the particular interval itself and a number of prior cardiac event intervals. For example, in one configuration, the HRI measurement for a current cardiac event interval is the average duration of the current cardiac event interval and the previous three cardiac event intervals. In this case, although the HRI measurement is calculated using several cardiac event intervals, the HRI measurement is considered to correspond to, or be associated with, only the current cardiac event interval.

At step 1304, the HRI measurement is compared to an HRI threshold to see if it satisfies the threshold. In the case where the HRI measurement is based on cardiac event intervals, the HRI threshold would be a time duration, below which possible exercise is indicated and above which no exercise is indicated. Alternatively, if the HRI measurement is in terms of beats-per-minute, the HRI threshold would be a number of beats-per-minute above which possible exercise is indicated and below which no exercise is indicated. The time duration or number of beats-per-minute may be patient specific and programmed into the device at implant and periodically updated, if necessary. For example, the HRI threshold may be between 0.500 and 0.750 seconds, or 80 to 120 beats-per-minute.

In the context of the present invention, when comparing a measurement to a threshold, the terms “exceeds” or “is greater than” can encompass instances when the measurement is equal to the threshold. Similarly, it is understood that the terms “falls below” or “is less than” a threshold can encompass instances when the measurement is equal to the threshold value. A person skilled in the relevant art will recognize that selection of a threshold, and how to treat the condition of equality between the threshold and the measurement, are design choices.

Continuing with step 1304, if the HRI measurement fails to satisfy the HRI threshold, no exercise is indicated and the process returns to step 1302. If, however, the HRI measurement does satisfy the HRI threshold, potential exercise is indicated, the time interval or duration of the cardiac event interval associated with the HRI measurements is recorded and the process proceeds to step 1306. In step 1306, subsequent HRI measurements are binned or counted as exercise or rest, in accordance with binning rules describe below.

During the binning process, HRI measurements are obtained and compared to the HRI threshold. A cardiac event interval having a HRI measurement that satisfies the threshold is binned or counted as exercise, while a cardiac event interval having a HRI measurement that does not satisfy the HRI threshold is binned or counted as rest. During this binning process, the time interval or duration of the cardiac event interval associated with the HRI measurements are recorded. As described below, these recorded intervals may be used to determine exercise duration. During the binning process, patient activity measurements may be obtained using an activity sensor. The activity measurement may be obtained by any suitable method. For example, the activity level may be determined by an accelerometer or a piezoelectric crystal. As described later, these measurements are recorded and used in conjunction with the above-described recorded intervals to determine exercise duration.

In another embodiment, the binning process is based on both the current HRI measurement and an average HRI measurement. The current HRI measurement is based on the current cardiac event interval. The average HRI measurement may be based on the average duration of a current cardiac event interval and a number of cardiac event intervals, including the current and prior cardiac event intervals. In this arrangement, a cardiac event interval is counted as exercise only if both its current HRI measurement and its average HRI measurement satisfy the HRI threshold. Likewise, a cardiac event interval is counted as rest only if both its current HRI measurement and its average HRI measurement fail to satisfy the HRI threshold. If neither of these conditions is satisfied, the cardiac event interval is not counted.

At step 1308, after each binning of a cardiac event interval, the total number of exercise counts is compared to an exercise-count threshold. The exercise-count threshold is a programmed value based on empirical data and may range from ten to sixty. In an exemplary configuration the number is sixty.

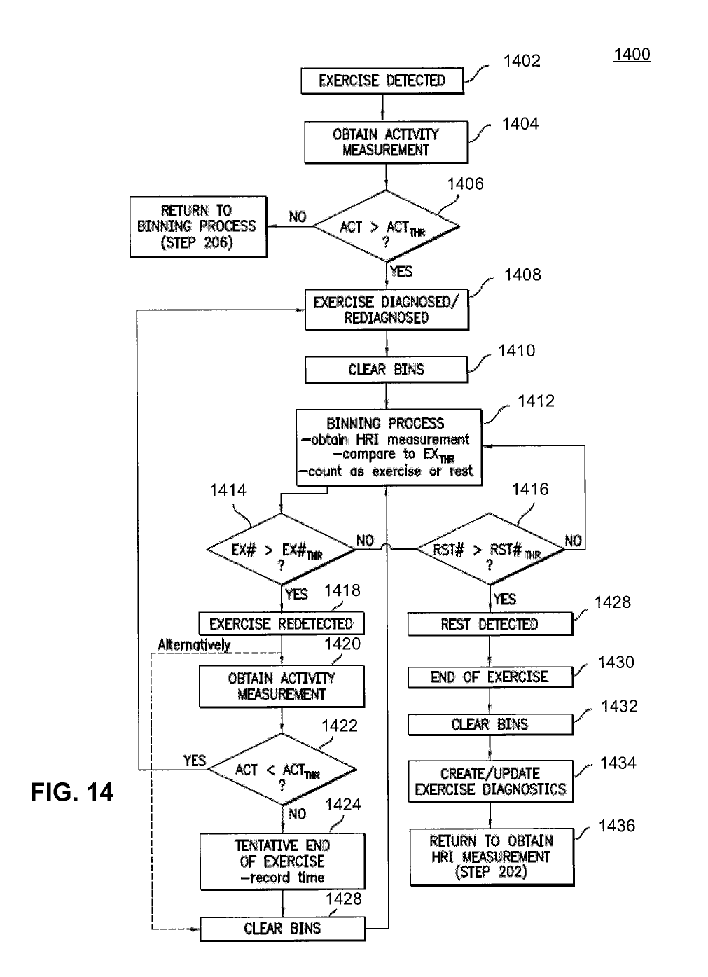

If, at step 1308, the exercise count satisfies its threshold, e.g., is “greater than” or is “greater than or equal to” the threshold, the process proceeds to step 1312, where exercise is “detected.” The process then precedes to steps 1314 and 1316 where the counting bins are cleared and the binning process (step 1306) is repeated. Alternatively, the process may proceed to consider patient activity measurements, as described below with respect to FIG. 14. Continuing with FIG. 13, if, as a result of the binning repeated process, the exercise count again satisfies the exercise-count threshold, exercise is “redetected.”

If at step 1308, the exercise count fails to satisfy the exercise-threshold count, e.g., is “less than” or is “less than or equal to” the threshold, the process proceeds to step 1310. At step 1310, the total number of rest counts is compared to a rest-count threshold. As with the exercise-count threshold, the rest-count threshold is a programmed value based on empirical data and may range from ten to sixty. In an exemplary configuration the number is sixty.

If, at step 1310, the rest count satisfies its threshold, e.g., is “greater than” or is “greater than or equal to” the threshold, the process proceeds to step 1318 where rest is “detected.” The process then proceeds to steps 1320 and 1322 where the counting bins are cleared and the process returns to its beginning, at step 1302. If the rest count does not satisfy the rest-count threshold, the process returns to step 1306 for further binning.

The process of FIG. 13 provides for the detection of exercise or rest of a patient based on heart rate data and a simple, low duty cycle binning process. As an enhancement to this process, patient activity data may be considered as a means of providing more definitive exercise diagnostics.

FIG. 14 is a flow chart illustrating an embodiment of a method 1400 for detecting exercise based on patient heart rate and activity data. Referring to FIG. 14, at step 1402 HRI measurements are monitored to detect exercise, as described above with respect to FIG. 13. Upon detection of exercise, at step 1404, the bins are cleared and an activity measurement is obtained. As with the previously described HRI measurements, an activity measurement may be a current measurement or may be a measurement based on an average of several activity measurements. The activity measurement obtained in step 1404 preferably corresponds to patient activity at the time of exercise detection and is thus based on one or more activity measurements stored during step 1306 of FIG. 13.

In step 1406, the activity measurement is compared with an activity threshold to see if it satisfies the threshold. The activity threshold may be patient specific and programmed into the device at implant and periodically updated, if necessary. For example, for an accelerometer sensor, the activity threshold, or accelerometer value, may be between five and twenty. If the activity measurement does not satisfy the activity threshold, e.g., is “less than” or “less than or equal to” the threshold, the process proceeds to step 1306 (FIG. 13) for further exercise binning.

If at step 1406 the activity measurement satisfies the activity threshold, e.g., is “greater than” or “greater than or equal to” the threshold, the process proceeds to steps 1408 and 1410 where exercise is “diagnosed” and the bins are cleared. It should be noted that the process draws a distinction between detection and diagnosis. Exercise is “detected” when the HRI measurements are such that the exercise count satisfies the exercise-count threshold. Exercise is “diagnosed” when both the exercise count satisfies the exercise-count threshold and the activity measurements satisfy the activity threshold. Thus, there can be no exercise diagnosis without exercise detection.

Returning to FIG. 14, at step 1412, HRI measurements are binned or counted as exercise or rest, in accordance with the binning rules described above with reference to FIG. 13. At step 1414, after each binning of a cardiac event interval, the total number of exercise counts is compared to an exercise-count threshold.

If, at step 1414, the exercise count satisfies its threshold, the process proceeds to step 1418, where exercise is “redetected.” The process may then proceed to steps 1420 and 1422, where activity measurements are obtained and compared to the activity threshold. If the activity measurement satisfies its threshold requirements, the process returns to step 1408 where exercise is “rediagnosed.” If, however, the activity measurement does not satisfy its threshold, the process proceeds to step 1424, where exercise is “tentatively ended.” The time of this tentative end of exercise is recorded. The process then proceeds to step 1426 where the bins are cleared and then returns to step 1412, where the HRI measurement binning process is repeated.

Alternatively, upon a redetection of exercise at step 1418, the process may bypass the activity measurement related steps 1420, 1422, 1424 and proceed directly to step 1426 wherein the count bins are cleared. In this scenario, activity measurements are used only for initial exercise diagnosis, at steps 1404 and 1406, thus conserving processor duty cycle.

If at step 1414, the exercise count fails to satisfy the exercise-count threshold, the process proceeds to step 1416, where the rest count is compared to the rest-count threshold. If, at step 1416, the rest count satisfies its threshold, the process precedes to steps 1428 and 1430 where rest is “detected” and exercise is “ended.” The process then proceeds to steps 1432, 1434 and 1436 where the counting bins are cleared, exercise diagnostics are created or updated and the process returns to its beginning, at step 1302 (FIG. 13). If the rest count does not satisfy its threshold, the process returns to step 1412 for further binning.

Graphs illustrative of example exercise periods that can be determined using the processes of FIGS. 13 and 14 are included in U.S. patent application Ser. No. 11/458,614, filed Jul. 19, 2006, entitled “Methods and Systems For Optimizing Exercise Compliance Diagnostic Parameters”; and U.S. patent application Ser. No. 11/561,267, filed Nov. 17, 2006, entitled “Methods and Devices for Monitoring Exercise Activity”, which was incorporated herein by reference above.

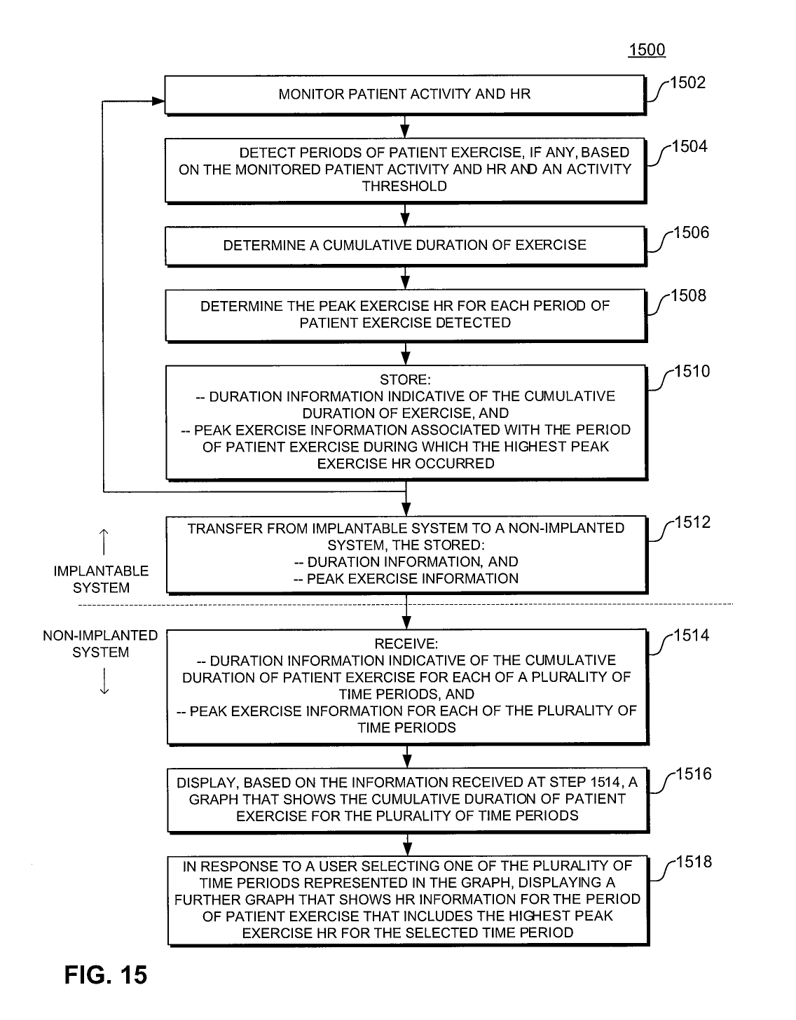

The flow chart of FIG. 15 will now be used to describe a method 1500 for patient monitoring, in accordance with an embodiment of the present invention. Referring to FIG. 15, steps 1502-1510 are performed by an implantable system (e.g., ICD 10) for each of a plurality of periods of time (e.g., a plurality of days, where each time period is one day); and steps 1514-1518 are performed by a non-implanted system (e.g., external device 102, or a system including external device 102).

At step 1502, patient activity and heart rate (HR) are monitored. Exemplary techniques for monitoring patient activity are described above with reference to step 202 of FIG. 2. HR can be monitored, e.g., in beats per minute, beats per second, or cardiac interval (e.g., R-R interval or P-P interval), or the like.

At step 1504, periods of patient exercise (if any), are detected based on the monitored patient activity and HR and an activity threshold. Exemplary techniques for determining an activity threshold are described above with reference to step 802 in FIG. 8. Exemplary techniques for detecting periods of patient exercise are described above, as well as below.

For example, exercise can be detected if a detected activity level exceeds an activity threshold and a HR measurement exceeds a corresponding threshold, as was described above with reference to steps 204 and 206 of FIG. 2. Alternatively, exercise can be detected in the manner described above with reference to FIG. 8. More specifically, correlation values can be determined based on the monitored patient activity and HR, e.g., as described above with reference to step 804. The correlation values can be compared to an activity threshold, e.g., as described above with reference to step 806. Entry to a period of exercise can be detected when the determined correlation values exceed the activity threshold for at least a predetermined amount of time (which may be referred to as an entry duration), e.g., as described above with reference to step 814. The exit (also referred as the end) of exercise can be detected when the determined correlation values do not exceed the activity threshold for at least a predetermined amount of time (which may be referred to as an exit duration), e.g., as was described above with reference to step 816. In another embodiment, exercise can be detected in the manner described with reference to FIGS. 13 and 14.

At step 1506, a cumulative duration of exercise for the time period (e.g., a day) is determined. This can include determining a duration of each period of patient exercise, and summing the durations to determine the cumulative duration of patient exercise for the time period. The duration of a period of patient exercise can be, e.g., the amount of time between the start (also referred to as entry) and the end (also referred to as exit) of exercise.

At step 1508, there is a determination of the peak exercise HR for each period of patient exercise detected. This can be accomplished, e.g., by simply determining the greatest HR (or shortest R-R or P-P interval) for each period of patient exercise detected.

At step 1510, duration information indicative of the cumulative duration of exercise for the time period (e.g., a day) is stored. Additionally, information is stored about the peak exercise associated with the period of patient exercise during which the highest peak exercise HR for the time period occurred. In accordance with an embodiment, the peak exercise information includes HR information.

If more the one period of patient exercise is detected during a time period (e.g., during a day), step 1510 can include identifying the highest peak exercise HR associated with the more than one period of patient exercise. Step 1510 can also include storing, as at least part of the peak exercise information, HR information for at least a substantial portion of the period of patient exercise that includes the highest peak exercise HR for the time period. For example, step 1510 can include storing HR information from the start (also referred to as entry) to the end (also referred to as exit) of the period of patient exercise that includes the highest peak exercise HR for the period of time (e.g., day).

As mentioned above, steps 1502-1510 occur for each of a plurality of periods of time. For example, steps 1502-1510 can be performed for each of 60 days.

At step 1512, the stored duration information and the stored peak exercise information (or information indicative thereof) are transferred from the implantable system (e.g., ICD 10) to a non-implanted system (e.g., external device 102). This enables, as described below, a graphical representation of the information to be displayed to a user. Such information can be transferred, e.g., after steps 1502-1510 are performed for the plurality of time periods (e.g., after 60 days). Alternatively, such transfer can occur more frequently, e.g., once per day, or once per week, or the like. In other words, such information can be from time to time uploaded to an external device (e.g., 102). Such an external device can be located, e.g., in the patients' home, and the information can be transmitted (e.g., through telephone lines or the Internet) to a medical facility where a physician can analyze the information. Alternatively, the external device can be located at a medical facility, and the information can be uploaded when the patient visits the facility. These are just a few examples, which are not meant to be limiting.

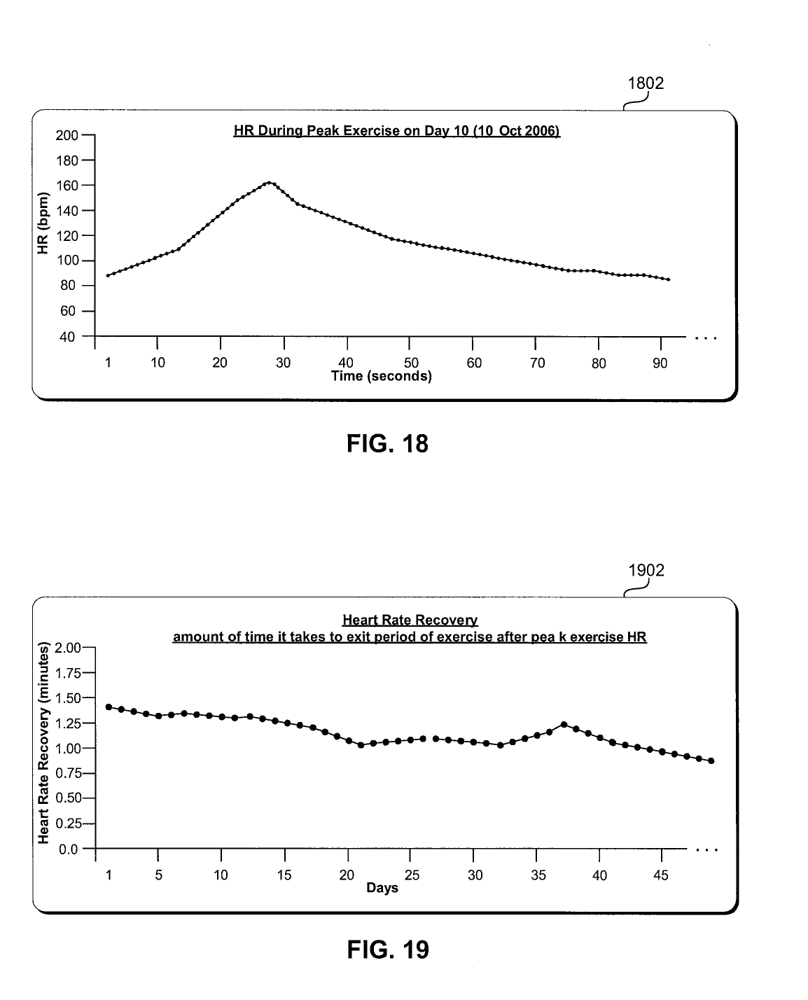

As specified at step 1514, the transferred information is received by the non-implanted system. After information is transferred to a non-implanted system, the information can be displayed to a user (e.g., doctor, nurse, clinician, etc.), in accordance with specific embodiments of the present invention, as specified at step 1516. In one embodiment, a graph that shows the cumulative duration of patient exercise for the plurality of time periods can be displayed. A graph 1702 in FIG. 17 is an example of such a graph. While the graph 1702 is a line graph, alternative types of graphs can be used to show the cumulative duration of patient exercise of the plurality of time periods, including, but not limited to, a bar graph. The data graphed in FIG. 17, as well as in FIGS. 18 and 19, are exemplary.

In accordance with an embodiment, a user is able to select one of the plurality of times represented in the graph, e.g., using a keyboard, mouse, joy stick, trackball, touchpad, or other pointing device, or, if the display screen is a touch screen, using the touch screen. As specified at step 1518, in response to a user selecting one of the plurality of time periods represented in the graph (e.g., graph 1702), a further graph is displayed, where the further graph shows HR information for the period of patient exercise that includes the peak patient exercise HR for the selected time period, preferably including HRs preceding and following the peak exercise HR. A graph 1802 in FIG. 18 is an example of such a further graph. While the graph 1802 is a line graph, alternative types of graphs can be used to show peak exercise HR information, including, but not limited to, a bar graph. The graph 1802 can partially or completely cover (i.e., overlay) the graph 1702, or a new page can be displayed that includes graph 1802. As shown in FIG. 18, the graph 1802 shows HR information that includes the highest peak exercise HR for the selected time period, including HRs preceding and following the peak exercise HR, and may include HRs from the entry to exit of exercise for the period of patient exercise that included the peak HR for the period of time (e.g., for the day).

Returning to FIG. 17, additionally, or alternatively, in response to a user selecting one of the plurality of time periods, a numerical value is displayed (e.g., in a pop-up block 1704) indicative of the cumulative duration of patient exercise for the selected time period. This enables that user to view information about exercise precisely, without having to visually follow a point on the graph all the way to the horizontal axis on the left. The user can make such selection, e.g., by moving a line 1706 (also referred to as a caliper) left or right, over one of the days. Similarly, in accordance with an embodiment, in response to a user selecting one of the plurality of time periods, a numerical value can be displayed (e.g., in a pop-up block similar to block 1704) indicative of the peak exercise HR and/or heart rate recovery for a selected time period, e.g., while FIGS. 18 and/or 19 are being displayed.

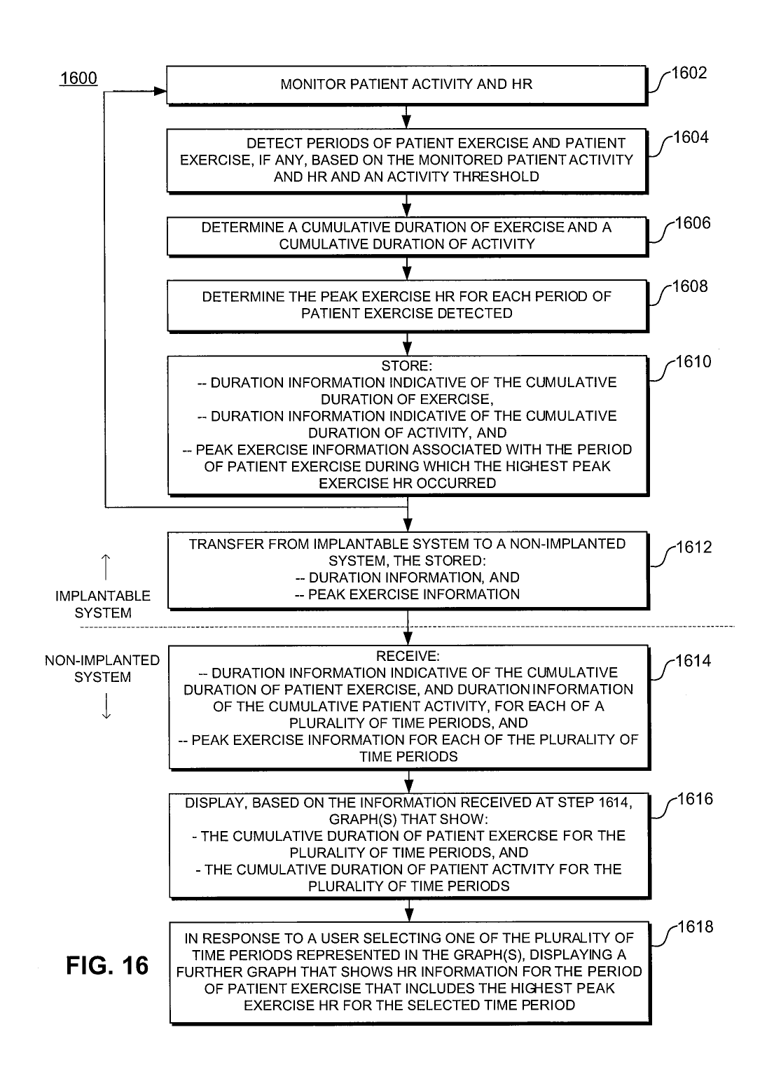

As will now be explained with reference to the high level flow diagram of FIG. 16, in accordance with specific embodiments of the present invention, periods of patient activity can also be detected for each of the plurality of periods of time (e.g., each of 60 days). Referring to FIG. 16, steps 1602-1610 are performed by an implantable system (e.g., ICD 10) for each of a plurality of periods of time (e.g., a plurality of days, where each time period is one day), in a similar manner as steps 1502-1510, as described above. Further, steps 1614-1618 are performed by a non-implanted system, as were steps 1514-1518 described above.

At step 1602, patient activity and HR are monitored, in the same manner as in step 1502. At step 1604, periods of patient exercise (if any), as well as periods of patient activity (if any) are detected based on the monitored patient activity and HR and an activity threshold. Exemplary techniques for detecting periods of patient exercise were described above, e.g., in the discussion of step 1504. Additionally, exemplary techniques for detection periods of activity were also described above, and are discussed below.

For example, activity can be detected if a detected activity level exceeds an activity threshold. In another embodiment, activity can be detected if a detected activity level exceeds an activity threshold and a HR measurement exceeds a corresponding threshold (e.g., HR>HR_{threshold}, or HRI<HRI_{threshold}), e.g., as was described above with reference to steps 204 and 206 of FIG. 2. Alternatively, activity can be detected in the manner described above with reference to FIG. 8. More specifically, correlation values can be determined based on the monitored patient activity and HR, e.g., as described above with reference to step 804. The correlation values can be compared to an activity threshold, e.g., as described above with reference to step 806. Activity can be considered to begin whenever the determined correlation value exceeds the activity threshold, as indicated at step 808. Activity can be considered to end when the determined correlation value no longer exceeds the activity threshold, as indicated at step 812. In accordance with an embodiment, entry to (i.e., the beginning of) a period of patient activity can be detected when N consecutive correlation values exceed the activity threshold, and (after entry to a period of patient activity has been detected) exit from (i.e., the end of) the period of patient activity can be detected when M consecutive correlation values do not exceed the activity threshold. In one embodiment N=M=1. In another embodiment, N=M=2. In other embodiments, N and M are not equal and/or N and/or M are greater than 2.

At step 1606, a cumulative duration of exercise for the time period (e.g., a day) is determined, as was described above with reference to step 1506. Additionally, at step 1606, a cumulative duration of activity for the time period (e.g., a day) is determined. This can include determining a duration of each period of patient activity, and summing the durations to determine the cumulative duration of patient activity for the time period. The duration of a period of patient activity can be, e.g., the amount of time between the start and the end of activity.

At step 1608, there is a determination of the peak exercise HR for each period of patient exercise detected, in the same manner as in step 1508.

At step 1610, duration information indicative of the cumulative duration of exercise for the time period is stored, in the same manner as in step 1510. Additionally, at step 1610, duration information indicative of the cumulative duration of activity for the time period is stored. Additionally, information is stored about the peak exercise associated with the period of patient exercise during which the highest peak exercise HR for the time period occurred. In accordance with an embodiment, the peak exercise information includes HR information.

As mentioned above, steps 1602-1610 occur for each of a plurality of periods of time. For example, steps 1602-1610 can be performed for each of 60 days.

At step 1612, the stored exercise and activity duration information and the stored peak exercise information (or information indicative thereof) are transferred from the implantable system (e.g., ICD 10) to a non-implanted system (e.g., external device 102), in a similar manner as was discussed above with reference to step 1512.

As specified at step 1614, the transferred information is received by the non-implanted system. After information is transferred to a non-implanted system, the information can be displayed to a user (e.g., doctor, nurse, clinician, etc.), in accordance with specific embodiments of the present invention, as specified at step 1616. In one embodiment, a graph that shows the cumulative duration of patient exercise for the plurality of time periods can be displayed (e.g., graph 1702 in FIG. 17), and a separate graph that shows the cumulative duration of patient activity for the plurality of time periods can be displayed in a separate graph (e.g., graph 1712 in FIG. 17). While the graph 1712 is a bar graph, alternative types of graphs can be used to show the cumulative duration of patient activity of the plurality of time periods, including, but not limited to, a line graph. It's also possible that a single graph shows both the cumulative duration of patient exercise, and the cumulative duration of patient exercise, for the plurality of time periods.

At step 1618, in response to a user selecting one of the plurality of time periods represented in the graph(s), a further graph is displayed, where the further graph shows HR information for the period of patient exercise that includes the peak patient exercise HR for the selected time period, preferably including HRs preceding and following the peak exercise HR, in the same manner as was described above with reference to step 1518. Additionally, or alternatively, in response to a user selecting one of the plurality of time periods, a numerical value is displayed (e.g., in a pop-up blocks 1704 and 1714) indicative of the cumulative duration of patient exercise and/or patient activity for the selected time period. This enables that user to view information about exercise and/or activity precisely, without having to visually follow a point on the graph all the way to the horizontal axis on the left. Again, the user can make such selection, e.g., by moving a line 1706 (also referred to as a caliper) left or right, over one of the days. Alternative techniques are also possible.

In accordance with certain embodiments, for each of the plurality of time periods, a heart rate recovery value can also be determined. The heart rate recovery values can be indicative of the amount of time it takes to exit the period of exercise after the peak exercise HR. In another embodiment, the heart rate recovery values can be indicative of how much HR falls within a specified amount of time following the peak exercise HR. In still another embodiment, the heart rate recovery values can be indicative of the amount of time it takes to reach a resting (e.g., baseline) HR after exiting the period of patient exercise. It's also possible that more than one of the various heart rate recovery values, or other heart rate recovery values, be determined. Such information can also be stored in the implantable device, and thereafter transferred to a non-implanted system so that a graphical representation of the heart rate recovery information can be displayed to a user.

Referring to FIG. 19, an exemplary heart rate recovery graph 1902 can, e.g., show the amount of time it takes to exit the period of exercise after the peak exercise HR, for each of the plurality of time periods. While the graph 1902 is a line graph, alternative types of graphs can be used, including, but not limited to, a bar graph or histogram. It's also possible that a single graph shows the information in FIG. 19 along with the information in FIGS. 17 and/or 18.

In one embodiment, the implantable device can store a histogram of heart rate recovery values. For every peak exercise stored, heart rate recover can be defined as the time from peak heart rate to exit from exercise (e.g., from the peak in FIG. 18 to when a correlation values drop below an activity threshold for an exit duration period). Heart rate recovery time can be logged for peak exercise per day. Peak heart rate during peak exercise can also be stored. Heart rate recovery time along with the peak heart rate information can then be displayed graphically to the user.

Based on the displayed information, a user (e.g., doctor, clinician, nurse, etc.) can assess a patient's cardiac condition and/or determine whether a patient has been following a recommended activity and/or exercise routine. For example, increases over time in duration of patient exercise and/or duration of patient activity can be interpreted as an improving HF condition. Decreases over time in duration of patient exercise and/or duration of patient activity can be interpreted as a worsening HF condition. Relatively no change over time in duration of patient exercise and/or duration of patient activity can be interpreted as relatively no change in HF condition.

Where heart rate recovery values are indicative of the amount of time it takes to exit the period of exercise after the peak exercise HR, decreases in such values can be interpreted as an improving HF condition, and increases in such values can be interpreted as a worsening HF condition. Where the heart rate recovery values are indicative of how much HR falls within a specified amount of time following the peak exercise HR, increases in such values can be interpreted as an improving HF condition, and decreases in such values can be interpreted as a worsening HF condition. Where the heart rate recovery values are indicative of the amount of time it takes to reach a resting HR after exiting the period of patient exercise, decreases in such values can be interpreted as an improving HF condition, and increases in such values can be interpreted as a worsening HF condition.

It will be appreciated by those skilled in the art that the above methods 200, 400, 500, 600, 700, 800, 900, 1000, 1300, 1400, 1500 and 1600 can be used within the hardware, software, and/or firmware of a pacing system, such as the ICD described earlier with reference to FIGS. 1A and 1B, for example. Such methods can also be used with implantable devices (e.g., monitors) that do not have pacing/or defibrillation capabilities.

Example embodiments of the methods, systems, and components of the present invention have been described herein. As noted elsewhere, these example embodiments have been described for illustrative purposes only, and are not limiting. Other embodiments are possible within the scope of the invention. Such embodiments will be apparent to persons skilled in the relevant art(s) based on the teachings contained herein. For example, while the activity/exercise duration method has been described as using certain data collection techniques, e.g., partitioning data acquisition periods into one or more sub-time periods, and data analyses techniques, e.g., histogram analysis, the methods, devices and systems are not limited to such techniques.

Furthermore, as another example, although the inventive methods are described with reference to an ICD, the methods are not limited to such a use. Illustratively, the inventive methods could be carried out with one or more external devices affixed to a patient's body to monitor heart rate and activity level, produce heart rate measurements and to determine exercise diagnostics such as, for example HRₘₐₓ, workload, heart rate recovery, activity duration and exercise duration. Illustratively, the patient may have affixed to their body a holter recording device to measure heart rate and an accelerometer to determine activity level.

Thus, the breadth and scope of the present invention should not be limited by any of the above-described exemplary embodiments, but should be defined only in accordance with the following claims and their equivalents.

1. A method for patient monitoring, comprising:
using an implantable system to perform steps (a) through (e) for each of a plurality of time periods
(a) monitoring patient activity and heart rate (HR);
(b) detecting periods of patient exercise, if any, based on the monitored patient activity and HR and an activity threshold;
(c) determining a cumulative duration of exercise for the time period;
(d) determining the peak exercise HR for each period of patient exercise detected; and
(e) storing duration information indicative of the cumulative duration of exercise for the time period, and storing peak exercise information associated with the period of patient exercise during which the highest peak exercise HR for the time period occurred, the peak exercise information including HR information; and
(f) transferring the stored duration information and the stored peak exercise information, or information indicative thereof, from the implantable system to a non-implantable system so that a graphical representation of the information can be displayed.

2. The method of claim 1, wherein the plurality of time periods comprise a plurality of days, and each time period comprises one day.

3. The method of claim 1, wherein step (b) includes for each of the plurality of time periods:
(b.1) determining correlation values based on the monitored patient activity and HR;
(b.2) comparing the correlation values to the activity threshold;
(b.3) detecting entry to a period of exercise when the determined correlation values exceed the activity threshold for at least an entry duration; and
(b.4) after entry to a period of exercise has been detected, detecting an exit from the period of exercise when the determined correlation values do not exceed the activity threshold for at least an exit duration.

4. The method of claim 1, wherein step (c) includes for each of the plurality of time periods:
(c.1) for each period of patient exercise, determining a duration of the period of patient exercise; and
(c.2) determining the cumulative duration of patient exercise for the time period by summing the durations determined at (c.1).

5. The method of claim 1, wherein step (e) includes for each of the plurality of time periods:
(e.1) if more the one period of patient exercise is detected during the time period, identifying the highest peak exercise HR associated with the more than one period of patient exercise; and
(e.2) storing, as at least part of the peak exercise information, HR information for at least a portion of the period of patient exercise that includes the highest peak exercise HR for the time period.

6. The method of claim 1, further comprising:
(g) displaying, based on the information transferred at step (f), a graph that shows the cumulative duration of patient exercise for the plurality of time periods.

7. The method of claim 6, wherein step (g) further includes, in response to a user selecting one of the plurality of time periods represented in the graph, a further graph that shows HR information for the period of patient exercise that includes the peak patient exercise HR for the selected time period, including HRs preceding and following the peak exercise HR.

8. The method of claim 6, further comprising, in response to a user selecting one of the plurality of time periods, displaying a numerical value indicative of the cumulative duration of patient exercise for the selected time period.

9. The method of claim 1, wherein:
step (b) also includes for each of the plurality of time periods, detecting periods of patient activity, if any, based on the monitored patient activity and HR and the activity threshold;
step (c) also includes for each of the plurality of time periods, determining a cumulative duration of activity; and
step (e) also includes for each of a plurality of time periods, storing, as part of the duration information, information indicative of the cumulative duration of activity for the time period.

10. The method of claim 9, wherein step (b) includes for each of the plurality of time periods:
(b.1) determining correlation values based on the monitored patient activity and HR;
(b.2) comparing the correlation values to the activity threshold; and
(b.3) detecting entry to a period of patient activity when N consecutive correlation values exceed the activity threshold; and
(b.4) after entry to a period of patient activity has been detected, detecting exit from the period of patient activity when M consecutive correlation values do not exceed the activity threshold.

11. The method of claim 9, wherein step (c) includes for each of the plurality of time periods:
(c.1) for each period of patient activity, determining a duration of the period of patient activity; and
(c.2) determining the cumulative duration of patient activity for the time period by summing the durations determined at (c.1).

12. The method of claim 9, further comprising:
(g) displaying, based on the information transferred at step (f), one or more graph that shows the cumulative duration of patient exercise for the plurality of time periods and the cumulative duration of patient activity for the plurality of time periods.

13. The method of claim 12, wherein step (g) further includes, in response to a user selecting one of the plurality of time periods represented in the one or more graph, displaying a further graph that shows HR information for the period of patient exercise that includes the highest peak exercise HR for the selected time period.

14. The method of claim 13, wherein the further graph displayed at step (g) shows HR information for the period of patient exercise that includes the highest peak exercise HR for time period, including HRs preceding and following the peak exercise HR.

15. The method of claim 12, further comprising, in response to a user selecting one of the plurality of time periods, displaying at least one of the following:
a numerical value indicative of the cumulative duration of patient exercise for the selected time period; and
a numerical value indicative of the cumulative duration of patient activity for the selected time period.

16. The method of claim 1, further comprising for each of the plurality of time periods, determining for the period of patient exercise that includes the highest peak exercise HR for the time period, a heart rate recovery value indicative of at least one of the following:
the amount of time it takes to exit the period of exercise after the peak exercise HR;
how much HR falls within a specified amount of time following the peak exercise HR; and
the amount of time it takes to reach a resting HR after exiting the period of patient exercise.

17. The method of claim 16, further comprising displaying, based on the information transferred at step (f), one or more graph that shows:
a heart rate recovery value for each of the plurality of time periods.

18. The method of claim 16, further comprising displaying, based on the information transferred at step (f), one or more graph that shows:
a heart rate recovery value for each of the plurality of time periods; and
peak exercise HR information for each of the plurality of time periods.

19. An implantable system for patient monitoring, comprising:
a patient activity monitor to monitor patient activity;
a heart rate monitor to monitor heart rate (HR);
an exercise monitor to detecting periods of patient exercise, if any, based on the monitored patient activity and HR and an activity threshold;
wherein the exercise monitor also determines a cumulative duration of exercise for each of a plurality of periods of time; and
wherein the exercise monitor also determines the peak exercise HR for each period of patient exercise detected; and
a memory to store duration information indicative of the cumulative duration of exercise for each of the plurality of periods of time, and to store peak exercise information associated with the period of patient exercise during which the highest peak exercise HR for each time period occurred, the peak exercise information including HR information; and
a transmitter to transfer the stored duration information and the stored peak exercise information, or information indicative thereof, from the implantable system to a non-implantable system so that a graphical representation of the information can be displayed.

20. A method for displaying information collected by an implantable system, the method comprising:
(a) receiving from an implantable system,
duration information indicative of the cumulative duration of patient exercise for each of a plurality of time periods, and
peak exercise information for each of the plurality of time periods, the peak exercise information including HR information associated with a period of patient exercise during which a highest peak exercise HR for the time period occurred;
(b) displaying, based on the information received at step (a), a graph that shows the cumulative duration of patient exercise for the plurality of time periods; and
(c) in response to a user selecting one of the plurality of time periods represented in the graph, displaying a further graph that shows HR information for the period of patient exercise that includes the highest peak exercise HR for the selected time period.

21. The method of claim 20, wherein the plurality of time periods comprise a plurality of days, and each time period comprises one day.

22. The method of claim 20, wherein the further graph displayed at step (c) shows HR information for at least a portion of the period of patient exercise that includes the highest peak exercise HR for the selected time period, including HRs preceding and following the peak exercise HR.

23. The method of claim 20, wherein:
step (a) also includes receiving as part of the duration information, information indicative of the cumulative duration of activity for each of the plurality of time periods; and
step (b) also includes displaying a graph that shows the cumulative duration of activity for the plurality of time periods.

24. The method of claim 20, wherein:
step (a) also includes receiving a heart rate recovery value, for each of the plurality of time periods, or information that enables a heart rate recovery value to be determined, wherein the heart rate recovery value is indicative of at least one of the following
the amount of time it takes to exit the period of patient exercise after the peak exercise HR;
how much HR falls within a specified amount of time following the peak exercise HR; and
the amount of time it takes to reach a resting HR after exiting the period of patient exercise; and
step (b) includes displaying a graph that shows the heart rate recovery value for the plurality of time periods.

25. An system for patient monitoring, comprising:
an implantable system including
a patient activity monitor to monitor patient activity;
a heart rate monitor to monitor heart rate (HR);
an exercise monitor to detecting periods of patient exercise, if any, based on the monitored patient activity and HR and an activity threshold;
wherein the exercise monitor also determines a cumulative duration of exercise for each of a plurality of periods of time; and
wherein the exercise monitor also determines the peak exercise HR for each period of patient exercise detected; and
a memory to store duration information indicative of the cumulative duration of exercise for each of the plurality of periods of time, and to store peak exercise information associated with the period of patient exercise during which the highest peak exercise HR for each time period occurred, the peak exercise information including HR information; and
a transmitter to transfer the stored duration information and the stored peak exercise information, or information indicative thereof, from the implantable system to a non-implantable system so that a graphical representation of the information can be displayed; and
a non-implantable system including
a receiver to receive the duration information and the stored peak exercise information, or information indicative thereof, from the implantable system; and
a display to display a graphical representation of the received information.
